(19) United States
(12) Patent Application Publication
(10) Pub. No.: US 2026/0204429 A1
(43) Pub. Date: Jul. 16, 2026

(54) Extracting An Entity-Specific Network From A Cross-Domain Network For Query Execution

(71) Applicant: Oracle International Corporation, Redwood Shores, CA (US)

(72) Inventors: Natalee Agassi, Blue Bell, PA (US); Amitabh Saikia, Mountain View, CA (US); Jigar Shirish Patel, Lenexa, KS (US); Raman Grover, Charlotte, NC (US); Vadim Khotilovich, Leawood, KS (US)

(73) Assignee: Oracle International Corporation, Redwood Shores, CA (US)

(21) Appl. No.: 19/398,616

(22) Filed: Nov. 24, 2025

Related U.S. Application Data
(60) Provisional application No. 63/745,461, filed on Jan. 15, 2025.

(51) Int. Cl. G16H 70/20; G16H 10/60; G16H 40/20

(52) U.S. Cl. CPC G16H 70/20; G16H 10/60; G16H 40/20

(57) ABSTRACT

Techniques for extracting an entity-specific network from a cross-domain network for query execution are disclosed. The cross-domain network includes multiple terminologies. Connections are generated (a) between terms, represented as nodes, within each terminology and (b) between terms of different terminologies to generate the cross-domain network. The connections are defined by relationships. An entity-specific network is a subset of the cross-domain network. The entity may be a symptom, a disease, or a condition. The system receives a query that includes a term. The system locates the node representing the term in the cross-domain network. The system identifies nodes within “N” hops of the node and generates an entity-specific network comprising the nodes within “N” hops of the node. The system executes the query on the entity-specific network. The query results are presented to the user on an interface, e.g., dashboard.

### BENEFIT CLAIMS; RELATED APPLICATIONS; INCORPORATION BY REFERENCE

This application claims the benefit of U.S. Provisional Patent Application 63/745,461, filed Jan. 15, 2025, that is hereby incorporated by reference.

The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to data management and query processing in distributed healthcare information systems. In particular, the present disclosure relates to systems and methods for extracting an entity-specific network from a cross-domain healthcare network to enable efficient and context-aware query execution across disparate clinical, administrative, and operational data sources.

### BACKGROUND

Healthcare organizations generate and maintain vast volumes of data across multiple domains, including electronic health records (EHRs), claims data, laboratory information systems, pharmacy dispensing systems, imaging repositories, public health registries, and payer databases. These domains often maintain independent data models, ontologies, and network relationships, which are represented through complex knowledge graphs or networked datasets comprising millions of interconnected entities and relationships.

The approaches described in this section are approaches that could be pursued but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIG. 2 illustrates an example set of operations for extracting an entity-specific network from a cross-domain network in accordance with one or more embodiments;

FIG. 3A illustrates an example embodiment of a cross-terminology network;

FIG. 3B illustrates an example embodiment of an entity-specific network derived from the cross-terminology network shown in FIG. 3A;

FIG. 4A illustrates a machine learning (ML) engine in accordance with one or more embodiments;

FIG. 4B illustrates an example set of operations of an ML engine in accordance with one or more embodiments; and

FIG. 5 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. DECISION SUPPORT TOOL SYSTEM ARCHITECTURE
3. EXTRACTING AN ENTITY-SPECIFIC NETWORK FROM A CROSS-DOMAIN NETWORK FOR QUERY EXECUTION
4. EXAMPLE EMBODIMENT
5. MACHINE LEARNING ARCHITECTURE
6. MACHINE LEARNING ENGINE OPERATIONS
7. GENERATIVE AI MODELS
8. PRACTICAL APPLICATIONS, ADVANTAGES, AND IMPROVEMENTS
9. HARDWARE OVERVIEW
10. MISCELLANEOUS; EXTENSIONS

### 1. General Overview

One or more embodiments extract an entity-specific network from a cross-domain network for query execution. The cross-domain network includes multiple terminologies, e.g., SNOMED CT, LOINC, MULTUM. In the cross-domain network, terms are represented by nodes. Connections are generated (a) between terms within each terminology and (b) between terms of different terminologies. The connections are defined by relationships. An entity-specific network is a subset of the cross-domain network. The entity may be a symptom, a disease, or a condition.

One or more embodiments receive a query that includes a target term. The system locates a target node representing the target term in the cross-domain network. The system identifies nodes within “N” hops of the target node and generates an entity-specific network comprising the nodes within “N” hops of the node. The system executes the query on the entity-specific network.

One or more embodiments apply a reasoner to the terms of the multiple terminologies to generate connections between nodes. The system may apply one or more ML models to the entity-specific network to select information for presenting to a user. An ML model may select a template for presenting information. Patient data may be compared to terms in the entity-specific network to identify overlap between the patient data and the entity-specific network.

One or more embodiments described in this Specification and/or recited in the claims may not be included in this General Overview section.

### 2. Decision Support Tool System Architecture

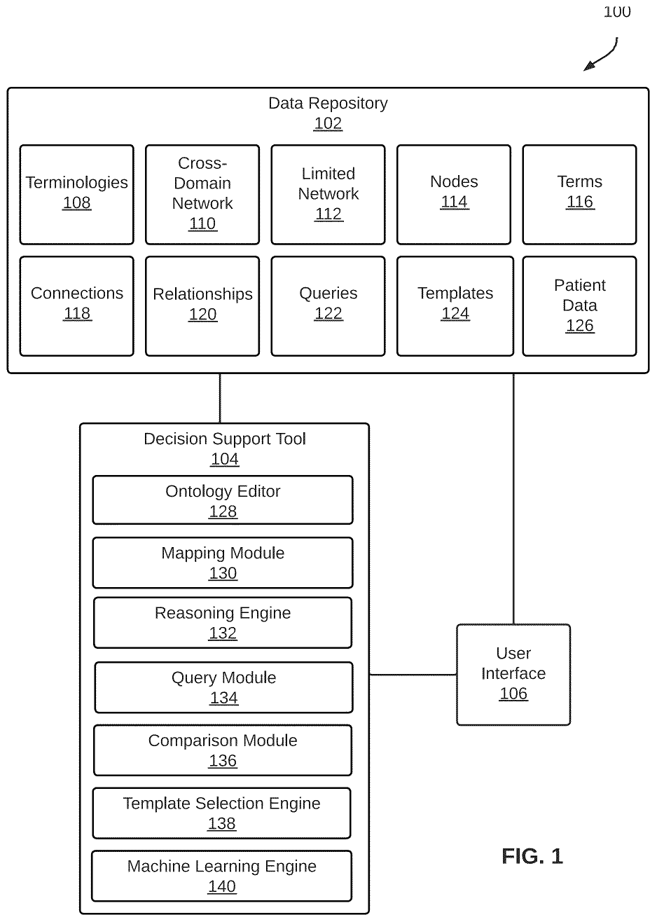

FIG. 1 illustrates a system 100 in accordance with one or more embodiments. As illustrated in FIG. 1, the system 100 includes a data repository 102, decision support tool 104, and an interface 106. The system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, a data repository 102 is any type of storage unit and/or device (e.g., a file system, database, collection of tables, or any other storage mechanism) for storing data. Furthermore, a data repository 102 may include multiple different storage units and/or devices. The multiple different storage units and/or devices may or may not be of the same type or located at the same physical site. Furthermore, a data repository 102 may be implemented or executed on the same computing system as the decision support tool 104 and the interface 106. Additionally, or alternatively, a data repository 102 may be implemented or executed on a computing system separate from the decision support tool 104 and the interface 106. The data repository 102 may be communicatively coupled with the decision support tool 104 and the interface 106 via a direct connection or via a network.

In one or more embodiments, the data repository 102 is populated with information from a variety of sources and/or systems. The data repository 102 may be populated with numerous data, such as terminologies 108, cross-terminology network 110, limited network 112, nodes 114, terms 116, connections 118, relationships 120, queries 122, templates 124, and patient data 126. Information describing mapping proprietary codes with standard codes using similarity between network relationships may be implemented across any of components within the system 100. However, this information is illustrated within the data repository 102 for purposes of clarity and explanation.

In one or more embodiments, terminologies 108 are used to ensure consistent communication, interoperability, and accurate documentation across healthcare systems. Terminologies 108 may include standard codes and proprietary codes. Standard codes are a set of industry or standardized codes that are widely adopted and used across the healthcare industry. Standard codes represent various aspects of patient care, procedures, diagnoses, medications, and other healthcare-related information. Proprietary codes are reference codes for clinical and/or non-clinical events or entities that are customized for consumers. When creating proprietary codes, local practice may be favored over uniformity of content, resulting in different consumers having unique sets of proprietary codes. Although the names of the proprietary codes may differ between consumers, many proprietary codes have semantic equivalences.

In one or more embodiments, terminologies 108 include International Classification of Diseases (ICD), Systematized Nomenclature of Medicine—Clinical Terms (SNOMED CT), Logical Observation Identifiers Names and Codes (LOINC), Current Procedural Terminology (CPT), RxNorm, North American Nursing Diagnosis Association International (NANDA-I), Healthcare Common Procedure Coding System (HCPCS), Diagnostic and Statistical Manual of Mental Disorders (DSM), and Anatomical Therapeutic Chemical Classification System (ATC). ICD, maintained by the World Health Organization (WHO), is for coding diseases, signs, symptoms, and procedures. SNOMED CT is a comprehensive, multilingual terminology for clinical documentation used to encode clinical concepts like diagnoses, symptoms, and procedures. SNOMED CT supports EHRs with over 300,000 clinical terms. LOINC standardize laboratory and clinical observations are used widely for lab results, clinical measurements, and other observations in electronic systems. CPT, maintained by the American Medical Association, includes standard codes for medical, surgical, and diagnostic procedures. RxNorm is a normalized naming system for generic and branded drugs maintained by the U.S. National Library of Medicine (NLM). RxNorm supports EHR systems in managing medication data and prescriptions. NANDA-I provides standard nursing diagnoses. HCPCS is comprised of codes for products, supplies, and services not included in CPT and is maintained by the Centers for Medicare & Medicaid Services (CMS). DSM is a classification of mental health disorders maintained by the American Psychiatric Association (APA) and used by clinicians and researchers to diagnose mental health conditions. OMAHA System is comprehensive terminology for documenting nursing practice and community health. ATC is maintained by the WHO and classifies drugs based on their therapeutic use and chemical properties.

In one or more embodiments, terminologies 108 include Unified Medical Language System (UMLS), Medical Dictionary for Regulatory Activities (MedDRA), International Classification of Functioning, Disability, and Health (ICF), International Union of Pure and Applied Chemistry (IUPAC), National Drug Code (NDC), Procedure Coding System (PCS), and Digital Imaging and Communications in Medicine (DICOM). UMLS, maintained by NLM, integrates various medical terminologies into a single framework for better data exchange. UMLS aides in mapping terms across different coding systems like ICD, SNOMED CT, and LOINC. MedDRA is standard terminology for adverse event reporting in clinical trials and pharmacovigilance maintained by the International Council for Harmonisation. ICF describes health conditions in terms of functioning and disability and is maintained by the WHO. MedDRA is used in rehabilitation, disability evaluation, and social care services. IUPAC Nomenclature provides chemical naming standards, including those for drug molecules. NDC, maintained by the U.S. Food and Drug Administration, identifies drugs in the United States. PCS, maintained by CMS, codes procedures performed in hospital settings (used alongside ICD). DICOM standardizes medical imaging data formats and communication protocols. DICOM is used in radiology and imaging systems to exchange and store images like MRIs, CT scans, and X-rays.

In one or more embodiments, terminologies 108 include Medi-Span, First Databank (FDB), Truven Micromedex, and UpToDate. Medi-Span, owned by Wolters Kluwer Health, includes drug databases with information on drug interactions, dosage, and safety. FDB, owned by Hearst Health, provides clinical drug knowledge and decision support. Truven Micromedex, owned by IBM Watson Health, formerly Truven Health Analytics, provides evidence-based drug, disease, and toxicology information. UpToDate, owned by Wolters Kluwer Health, provides clinical decision support resource offering evidence-based guidelines.

In one or more embodiments, terminologies 108 include Code Set 72. Code Set 72, also known as Cerner Clinical Event Codes, is a proprietary code set maintained by Cerner Corporation. Code Set 72 is an extensive collection of codes used to represent various clinical and non-clinical events, including clinical documents, note types, immunizations, and clinical observations, such as laboratory results and vital signs. Code Set 72 is highly customized by Cerner clients, and the specific codes used may vary depending on the client's healthcare system. The general structure and purpose of the code set remains consistent across Cerner clients. Code Set 72 is a very large code set, encompassing a wide range of clinical events. The specific codes used in Code Set 72 are tailored to meet the specific needs of each Cerner client.

In one or more embodiments, terminologies 108 include Multum, a healthcare database that offers detailed drug information to support clinical decision-making. Multum is widely integrated into systems, like EHRs and consumer drug resources. Multum provides critical data for medications, including drug interactions, dosages, and therapeutic uses, aimed at promoting safe medication practices. Managed by Oracle Health (formerly Cerner), Multum offers several tools tailored to healthcare organizations, including Lexicon Plus, which supplies comprehensive drug and disease nomenclature, and VantageRx, which delivers drug knowledge through a structured database format. The database is frequently used in clinical environments to improve prescribing accuracy and avoid adverse drug events by highlighting interactions and warnings. Multum data supports software development kits (SDKs), e.g., addVantageRx and SubscribeRx, which allow organizations to embed or integrate drug content directly into their applications. This makes it an essential component in both consumer-oriented drug information portals and provider-based medication management systems.

In one or more embodiments, cross-terminology or cross-domain network 110 is an ontological structure or framework that connects and maps concepts across multiple terminologies. Cross-terminology network 110 allows for seamless translation and interoperability between different coding systems used in healthcare. Cross-terminology network 110 forms a web of linked nodes from various terminologies, facilitating efficient information sharing and retrieval across platforms that use different coding systems.

In one or more embodiments, limited network 112 is an entity-specific network or subnetwork of the cross-terminology network 110. Limited network 112 is an extracted from the cross-terminology network 110. Limited network 112 includes an entity, i.e., a target term represented by a target node, and all terms and/or relationship within “N” number of hops, e.g., 4, 5, 6, from the target node. Hops represent connections between nodes that extend across terminologies or may be within the same terminology. The farther away a node is from the target node, the less relevant the term and/or relationships represented by the nodes are to the target term.

In one or more embodiments, nodes 114 represent individual entities or concepts (e.g., conditions, medications, procedures, or patient data elements) within cross-terminology network 110. Each node serves as a point of reference or a unique data unit labeled with a term, i.e., a diagnosis code or medication name, and nodes are interconnected through relationships to form a structured web of healthcare knowledge.

In one or more embodiments, terms 116 are standardized labels used to describe specific concepts within a set of terminology, e.g., diseases, medications, procedures, or symptoms. Each term corresponds to a unique code in a set of terminologies and ensures consistency and precision in data documentation and retrieval.

In one or more embodiments, connections 118 are links between nodes 114 that define how different medical entities relate to one another. Connections 118 represent relationships. By connecting concepts, e.g., diagnoses, treatments, and symptoms, connections 118 facilitate a comprehensive understanding of complex health data, supporting interoperability, clinical decision support, and efficient data retrieval across various healthcare systems.

In one or more embodiments, relationships 120 define the type and nature of connections between different concepts or nodes 114, e.g., diagnoses, treatments, and symptoms. Common relationships include, “is-a,” e.g., “Type 1 Diabetes is-a Diabetes,” “treats,” e.g., “Insulin treats Diabetes”), and “part-of,” e.g., “Heart is part-of Cardiovascular System”. Relationships 120 organize medical data into meaningful structures, enabling systems to interpret complex medical knowledge, support decision-making, and allow data interoperability across healthcare platforms.

In one or more embodiments, queries 122 refer to user input requesting information from the limited network 112. Queries 122 include at least one entity, e.g., symptom, disease, and condition. Queries 122 may be patient specific, population specific, or all inclusive. Queries 122 may be generated and executed in real time. Queries 122 may be constructed using structured query language or other query language.

In one or more embodiments, templates 124 are layouts designed to present insights derived from limited network 112 effectively. Templates 124 can be customized based on the user, e.g., clinicians, researchers, patients, and the nature of the query. Templates 124 may (a) provide high-level insights for quick understanding, (b) be used to visualize the relationships within the limited network 112, (c) provide detailed analysis of the extracted subnetwork, (d) highlight relationships across specific domains, (e) present dynamic or drill-down information, and (f) communicate findings. Templates 124 ensure uniform reporting across different queries or cases, simplify communication, provide tailored information, and are adaptable to various domains.

In one or more embodiments, patient data 126 refers to any information about a patient that is collected during the course of the healthcare of the patient. Patient data 126 may be received from a variety of sources and may include a wide range of information types. Patient data 126 may include demographic data, e.g., age, gender, ethnicity, address, and contact details; medical history, e.g., records of past medical conditions, surgeries, treatments, and hospitalizations; clinical data, e.g., information obtained from medical examinations, including physical exams, vital signs, and symptoms; diagnostic data, e.g., results from laboratory tests (blood tests, urine tests, etc.), imaging studies (X-rays, MRIs, CT scans), and pathology reports; and, medication data, e.g., details about current and past medications, including dosages, frequency, and any adverse reactions or allergies. Patient data 126 may also include treatment data, e.g., information about ongoing treatments, including surgeries, therapies, and other interventions; progress notes, e.g., notes made by healthcare providers documenting patient encounters, observations, and treatment plans; administrative data, e.g., information related to healthcare administration, such as insurance details, billing records, and appointment schedules; behavioral data, e.g., information about lifestyle factors, such as smoking status, alcohol consumption, diet, and exercise habits; patient-generated data, e.g., data collected directly from patients, such as through surveys, wearable devices, or home monitoring equipment.

In one or more embodiments, patient data 126 may be received from EHRs, personal health records (PHRs), laboratory information systems (LIS), radiology information systems (RIS), picture archiving and communication systems (PACS), pharmacy information systems (PIS), wearable devices and remote monitoring systems, patient portals, insurance claim data, and Health Information Exchanges (HIEs). EHRs are digital versions of patients'paper charts that provide real-time, patient-centered records accessible to authorized healthcare providers. PHRs are health records maintained by patients themselves, often through digital platforms or mobile apps. LIS are systems that manage lab test orders and results, integrating with EHRs and other hospital information systems. RIS and PACS are systems that manage radiological records and imaging data. PIS are systems that manage medication orders, dispensing, and inventory in healthcare settings. Wearable devices and remote monitoring systems include various devices, such as fitness trackers, heart rate monitors, and glucose monitors, that collect health data outside traditional healthcare settings. Patient portals are online platforms that provide patients with access to their health information, appointment scheduling, and communication with healthcare providers. Insurance claims data is data generated from insurance claims that provide information on diagnoses, treatments, and healthcare utilization. HIEs are networks that enable the sharing of health information across different healthcare organizations and systems.

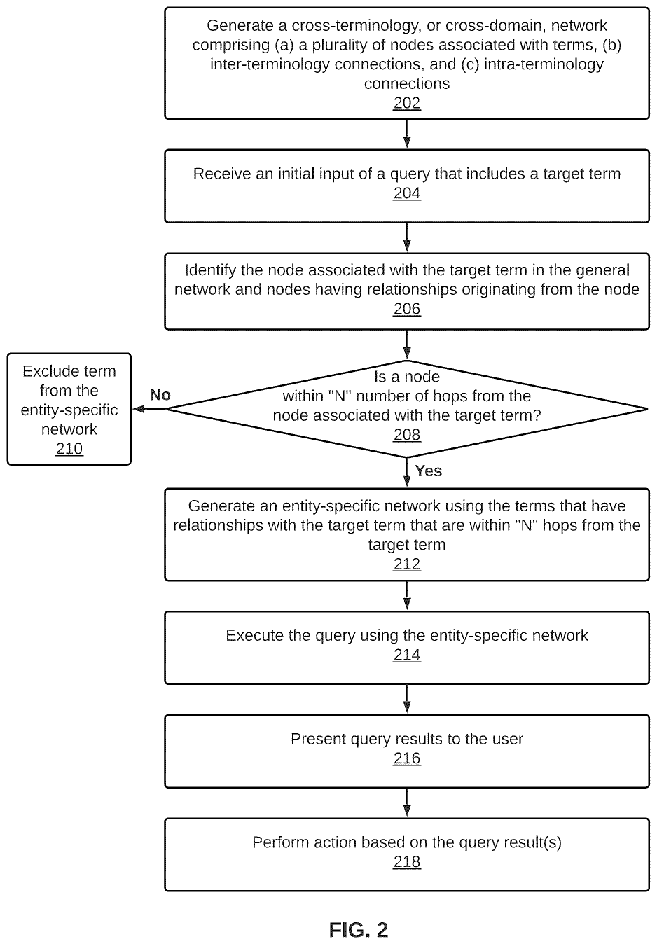

In one or more embodiments, decision support tool 104 refers to hardware and/or software configured to perform operations described herein for mapping proprietary codes with standard codes using similarity between network relationships. Examples of operations for mapping proprietary codes with standard codes using similarity between network relationships are described below with reference to FIG. 2.

In an embodiment, decision support tool 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, decision support tool 104 includes an ontology editor 128. Ontology editor 128 is software and/or hardware configured to perform operations described for creating, modifying, and managing ontologies, i.e., formal representations of knowledge in a specific domain. Ontology editors enable users to define concepts, specify relationships between the concepts, and organize information hierarchically. Ontology editor 128 is used to build and maintain knowledge models, e.g., cross-terminology network 110, supporting tasks like data integration, semantic search, and decision-making.

In one or more embodiments, ontology editor 128 includes one or more of Protégé, TopBraid Composer, OntoStudio, WebProtege, NeOn Toolkit, and/or Visual Notation for OWL Ontologies (VOWL). Protégé is an open-source ontology editor that supports the creation, visualization, and management of ontologies. Protégé provides tools for defining classes, properties, and relationships, and it supports the Web Ontology Language (OWL) standard. TopBraid Composer is a comprehensive tool for developing, testing, and managing semantic models, including ontologies. TopBraid Compser supports various standards, like OWL, RDF, and SPARQL, and provides a visual interface to build and edit ontologies. OntoStudio is an ontology editor for developing OWL and RDF ontologies. OntoStudio supports advanced features, like reasoning and ontology validation, and includes tools for graphical modeling and API integration. WebProtege is a web-based version of Protégé that allows users to collaboratively create, edit, and share ontologies online. NeOn Toolkit is an open-source, extensible toolkit for building ontologies and semantic web applications. NeOn Toolkit offers support for multi-ontology development and reasoning. VOWL is visual editor designed to create and understand OWL ontologies.

In one or more embodiments, decision support tool 104 includes a mapping module 130. The mapping module 130 is software and/or hardware configured to perform the operations described for translating and aligning data between different formats, schemas, or terminologies. Mapping module 130 connects disparate healthcare terminologies (e.g., SNOMED CT, ICD-10, LOINC, and RxNorm) that allow systems to communicate and share data accurately. Mapping module 130 may use tools (e.g., UMLS Metathesaurus, BioPortal, or OMOP) that have pre-existing mappings between common healthcare terminologies. By establishing equivalences and relationships between terms across the terminologies, mapping module 130 supports interoperability, ensuring that clinical data remains consistent and usable across various healthcare platforms and applications.

In one or more embodiments, decision support tool 104 includes a reasoning engine 132. Reasoning engine 132 includes software and/or hardware configured to perform the operation described herein for applying logical rules and algorithms to terminologies to infer new information, validate data relationships, and/or make decisions based on predefined knowledge.

In one or more embodiments, reasoning engine 132 includes one or more of the following: Hermit, FaCT++, Pellet, RacerPro, Snorocket, ELK, and PROTON. Hermit is an OWL reasoner that classifies and checks the consistency of ontologies. FaCT++ is a description logic reasoner optimized for handling complex ontologies, especially those in OWL DL. Pellet is a well-known, open-source OWL DL reasoner that supports SPARQL queries, rule-based reasoning, and consistency checking. RacerPro is a high-performance reasoner for OWL and RDF that supports both standard and complex reasoning tasks. Snorocket is a scalable ontology classifier that efficiently processes large medical terminologies, such as SNOMED CT, and is commonly used in healthcare due to its focus on speed and scalability. ELK is a highly efficient reasoner for OWL 2 EL. PROTON is a rule-based reasoning engine designed to work with ontologies and semantic models that enables advanced query capabilities.

In one or more embodiments, query module 134 is software and/or hardware configured to perform the operations described herein for receiving a query from a user and executing the query. Query module 134 extracts, analyzes, and presents information from the entity-specific networks. Query module 134 may incorporate generative models to process, interpret and execute queries.

In one or more embodiments, comparison module 136 is software and/or hardware configured to perform the operations described herein for comparing patient data and a limited network. The comparison module 136 identifies similarities, discrepancies, or risks based on the relationships and patterns within the subnetwork.

In one or more embodiments, template selection module 138 is software and/or hardware configured to perform operations described herein for selecting the most appropriate template for presenting information from a limited network. Template selection module 138 is designed to enhance the efficiency and clarity of data communication by aligning output formats with the needs and context of the user.

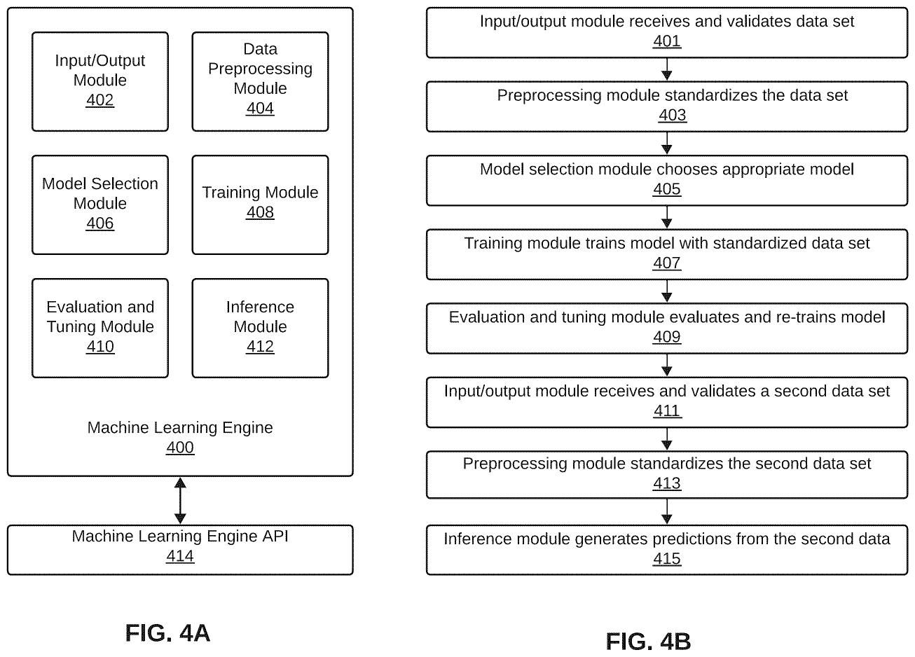

In one or more embodiments, decision support tool 104 includes a machine learning engine 140. Machine learning engine 140 will be described below with reference to FIG. 4B.

In one or more embodiments, interface 106 refers to hardware and/or software configured to facilitate communications between a user and decision support tool 104. Interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of interface 106 are specified in different languages. The behavior of user interface elements is specified in a dynamic programming language such as JavaScript. The content of user interface elements is specified in a markup language, such as hypertext markup language (HTML) or XML User Interface Language (XUL). The layout of user interface elements is specified in a style sheet language such as Cascading Style Sheets (CSS). Alternatively, interface 106 is specified in one or more other languages, such as Java, C, or C++.

### 3. Extracting an Entity-Specific Network From a Cross-Domain Network for Query Execution

FIG. 2 illustrates an example set of operations for extracting an entity-specific network from a cross-domain network in accordance with one or more embodiments. One or more operations illustrated in FIG. 2 may be modified, rearranged, or omitted. Accordingly, the particular sequence of operations illustrated in FIG. 2 should not be construed as limiting the scope of one or more embodiments.

One or more embodiments generate a cross-terminology, or general, network that includes (a) a plurality of nodes associated with terms, (b) inter-terminology connections, and (c) intra-terminology connections (Operation 202). The cross-terminology network provides a unified graph-based representation that models semantic relationships across multiple medical coding systems and terminologies. Each node in the network represents a standardized or proprietary term (e.g., a code, label, or description), and each connection represents a semantic or hierarchical relationship between two or more terms. The system may use the cross-terminology network to translate, align, and/or reason over disparate healthcare vocabularies, thereby improving interoperability between heterogeneous datasets.

In one or more embodiments, the system initially receives one or more user selections that specify the plurality of terminologies to be incorporated into the cross-terminology network. These terminologies may correspond to one or more data domains or clinical contexts of interest. For example, diagnosis-related terminologies may include ICD-10-CM, SNOMED CT, or MedDRA; laboratory test terminologies may include LOINC or UCUM; and medication terminologies may include RxNorm, Multum, or NDC. The user selections may be entered via a graphical configuration interface or retrieved automatically based on the type of dataset or query being processed. The greater the number of selected terminologies, the greater the potential number of inter-terminology and intra-terminology relationships that can be represented in the resulting graph.

In one or more embodiments, the system accesses the selected terminologies from one or more data sources. Terminologies may be accessed through public application programming interfaces (APIs) (e.g., FHIR Terminology Service, UMLS API), institutional repositories, or terminology provider websites. Certain terminologies, such as SNOMED CT, LOINC, or RxNorm, make formal ontology files available in machine-readable formats, such as CSV, JSON, OWL (Web Ontology Language), or RDF (Resource Description Framework). Upon access, the system parses the ontology data, extracting term identifiers, preferred names, synonyms, and relationships, and loads the structured data into a terminology ingestion module or directly into an ontology editor for integration into the cross-terminology network.

In one or more embodiments, the ontology editor provides a visualization and authoring environment for creating and refining the cross-terminology network. Within the editor, each imported terminology forms an initial subgraph of intra-terminology connections (e.g., parent-child or broader-narrower hierarchies inherent to that terminology). The ontology editor allows creation of inter-terminology connections between nodes originating from different terminologies. These connections may be defined as object properties or semantic relationships, such as equivalentTo, broaderThan, narrowerThan, relatedTo, or associatedWith.

In an example, a node representing “Hemoglobin A1c [Mass/volume] in Blood (LOINC:4548-4)” may be connected via a relatedTo relationship to “Glycated Hemoglobin Measurement (SNOMED CT:43396009).” Similarly, “Type 2 Diabetes Mellitus (SNOMED CT:44054006)” may be linked to “E11—Type 2 Diabetes Mellitus (ICD-10)” via an equivalentTo property. Each relationship may include metadata attributes, such as the mapping source, provenance, confidence score, and version identifiers.

In one or more embodiments, the ontology editor incorporates automated mapping features that leverage pre-existing crosswalks, such as mappings from UMLS Concept Unique Identifiers (CUIs), NLM LOINC-SNOMED mappings, or RxNorm-Multum correspondence tables. The system may apply string similarity, lexical matching, and/or vector embedding similarity techniques to discover candidate alignments between terms not already covered by existing mappings. These automated or semi-automated mappings enable more efficient population of the inter-terminology connections within the cross-domain network.

In one or more embodiments, the system integrates a reasoning engine with the ontology editor to ensure logical consistency and semantic completeness of the cross-terminology network. The reasoning engine may employ description logic and ontological inference techniques (e.g., OWL reasoners, such as Pellet or HermiT) to validate defined relationships, identify duplicate or contradictory connections, and infer new indirect relationships based on transitivity or equivalence rules. For instance, if Term A (ICD-10) is equivalentTo Term B (SNOMED CT), and Term B is broaderThan Term C (LOINC), the reasoning engine may infer that Term A is broaderThan Term C.

In one or more embodiments, the reasoning process allows the system to expand the cross-domain network beyond explicitly defined mappings, enriching the network with inferred connections that capture semantic proximity across terminologies. The engine may output a consolidated, validated graph structure comprising (a) intra-terminology relationships representing native hierarchies and (b) inter-terminology relationships representing cross-domain alignments. The system may store the resulting graph in a graph database or RDF triplestore, where nodes correspond to individual terms and edges correspond to semantic relationships. The cross-terminology network provides a unified, machine-interpretable representation of medical knowledge spanning multiple terminologies. The cross-terminology network forms a foundational data layer that supports downstream operations, such as entity-specific network extraction, semantic query expansion, terminology harmonization, and cross-domain reasoning for healthcare data integration and analytics.

One or more embodiments receive an initial input of a query that includes a target term (Operation 204). The query represents a user request for information, relationships, and/or insights concerning a clinical entity of interest. A user may provide the query through a GUI or dashboard associated with the cross-terminology system. The system may receive the query as natural language text, a structured query, or a formal query expression, depending on the technical proficiency of the user and the interface mode.

In an example, a clinician may type “Show related lab tests for diabetes” in a natural-language input field, or alternatively, may specify a structured query such as {entity: “diabetes,” type: “lab_test”}. In another example, an analytics engine or third-party application may generate the query programmatically via an API call. Regardless of the source, the system may extract a target term from the query corresponding to the entity or concept of interest. The target term may include a symptom, disease, medication, procedure, laboratory test, or any other concept recognizable within the cross-terminology network.

In one or more embodiments, the system parses the query and converts the user input into a machine-readable query representation. The parsing process may involve multiple layers of linguistic and semantic analysis, including tokenization, part-of-speech tagging, entity recognition, and intent classification. The system recognizes key entities, such as medications, conditions, procedures, or laboratory tests, and identifies different intents, such as “retrieve related drugs,” “compare outcomes,” or “find equivalent codes.” Contextual elements may also be extracted to refine the scope of the query, e.g., “focus on drug-to-drug interactions,” “within pediatric population,” or “according to 2025 version of ICD-10.”

In one or more embodiments, the system employs ML models, e.g., a named entity recognition (NER) model, transformer-based language model (e.g., BioBERT, SapBERT, or ClinicalBERT), and/or an ontology-aware parser, to perform semantic parsing and intent extraction. The system may transform the parsed output into a structured intermediate representation, such as a semantic query graph or abstract syntax tree (AST). In a structured intermediate representation, a node represents an identified entity or attribute, and each edge represents a relationship or dependency inferred from the user's query.

In one or more embodiments, the system performs term disambiguation to resolve cases where a token or phrase may refer to multiple possible meanings. For example, the abbreviation “DM” may represent “Diabetes Mellitus” in a clinical context or may denote “Data Management” or “Dermatomyositis” in other contexts. To resolve such ambiguities, the system may analyze contextual signals (e.g., surrounding words, user role, domain of the dashboard, or recent query history) to determine the most probable meaning based on a contextual similarity score or domain-specific embedding model.

In one or more embodiments, the system leverages the cross-terminology network to support disambiguation. For instance, when the query term matches multiple nodes across terminologies, the system may use network topology, such as node degree, relationship density, or prior usage frequency, to select the most contextually relevant node as the target. Additionally, the system may apply vector similarity computations between the embedding of the ambiguous term and candidate nodes'embeddings within the cross-terminology graph to select the most semantically aligned entity.

In one or more embodiments, after disambiguation, the system constructs a normalized, machine-interpretable query that references the canonical identifiers of the recognized entities within the cross-terminology network. The normalized query can then be executed against the cross-terminology network to extract related terms, relationships, or entity-specific networks, depending on the identified user intent. By converting the user's free-form or structured query into a machine-readable representation anchored to standard terminology identifiers, the system enables consistent, context-aware access to cross-terminology relationships. This facilitates more accurate retrieval of relevant data elements, mappings, and/or knowledge across heterogeneous medical terminologies and domains.

One or more embodiments identify the node associated with the target term in the general network and nodes having relationships originating from the node (Operation 206). The system first determines, within the cross-terminology network, the target node corresponding to the target term extracted from the query. The system may represent the cross-terminology network as a graph data structure, e.g., an adjacency list or adjacency matrix. Nodes in the graph data structure correspond to terms from various terminologies (e.g., ICD-10, SNOMED CT, LOINC, RxNorm), and edges represent semantic relationships (e.g., equivalentTo, broaderThan, relatedTo). Each edge may include metadata, such as relationship type, weight, source terminology, provenance, confidence score, and creation timestamp.

In one or more embodiments, upon locating the target node, the system retrieves outgoing and incoming edges connected to that node. These edges collectively define the neighborhood of the target node, representing other nodes that maintain direct semantic relationships with the target concept. For example, if the target node represents “Type 2 Diabetes Mellitus (SNOMED CT:44054006),” the system may identify linked nodes, such as “E11—Type 2 Diabetes Mellitus (ICD-10)” (equivalentTo), “Metformin (RxNorm:86009)” (relatedTo), and “HbA1c Measurement (LOINC:4548-4)” (associatedWith). The directly connected nodes, identified through the extracted edges, form a first-level neighborhood, also referred to as Level-1 nodes or 1-hop nodes. Each Level-1 node lies one edge (or one hop) away from the target node. The relationships between the target node and each Level-1 node constitute first-order relationships, such as equivalence, association, and/or hierarchical linkage.

In one or more embodiments, the system performs a graph traversal operation to identify additional layers of connected nodes. Nodes connected to the first-level nodes, but not already part of the set of previously identified nodes, form a second level of nodes (Level-2 nodes), representing entities located two hops from the target node. Similarly, nodes connected to the second-level nodes, but not yet included in the first two levels, form a third level of nodes (Level-3 nodes), located three hops from the target node. This recursive traversal can continue to an arbitrary or system-defined depth, allowing the formation of additional levels (Level-n) as needed to satisfy query depth, confidence thresholds, and/or semantic coverage requirements.

In one or more embodiments, the traversal process is implemented using a Breadth-First Search (BFS) or Depth-Limited Search algorithm executed on the cross-terminology graph structure. The system may expand each iteration one level outward from the target node and record discovered nodes and their connecting relationships in a temporary sub-graph structure. The system may weight base traversals by relationship types or confidence scores, e.g., prioritizing equivalentTo and broaderThan relationships over relatedTo or associatedWith, so high-reliability relationships are expanded first.

In one or more embodiments, the system applies filtering criteria or hop limits to control network expansion. For example, the traversal depth may be capped at three hops to prevent excessive graph growth or to maintain semantic relevance. Alternatively, the traversal may terminate when the cumulative relationship confidence drops below a threshold or when the number of new nodes discovered in a subsequent level falls below a minimum count. Such dynamic termination rules ensure computational efficiency while preserving meaningful semantic coverage. The system may store each node and/or edge discovered through the traversal as part of an entity-specific network data structure. The system may represent the subnetwork in a graph database (e.g., Neo4j, Amazon Neptune, or an RDF triplestore), in an in-memory knowledge graph, and/or as a serialized JSON-LD graph object. The resulting subnetwork encapsulates relevant semantic relationships centered around the target term, including both direct (1-hop) and indirect (multi-hop) relationships.

In one or more embodiments, the system assigns level identifiers or hop indices to nodes within the subnetwork, enabling hierarchical visualization and controlled reasoning. For example, a visualization module may render the target node at the center of a radial graph, with Level-1, Level-2, and Level-3 nodes arranged concentrically outward to illustrate semantic proximity. The hierarchical structure allows the user or downstream analytics components to differentiate between core relationships (e.g., direct equivalents) and contextual relationships (e.g., related laboratory observations or medications).

In one or more embodiments, the system computes aggregate metrics across levels, such as node centrality, edge density, and/or concept diversity, to characterize the strength or breadth of the entity's semantic context. The system may use these metrics to further refine or rank the relevance of nodes in downstream applications, such as query expansion, terminology alignment, and/or clinical reasoning.

One or more embodiments determine if a node is within an “N” number of hops from the node associated with the target term (Operation 208). The system analyzes the previously traversed cross-terminology graph to identify nodes located within a specified proximity threshold, where proximity is defined in terms of graph-theoretic distance, i.e., the number of edges connecting any given node to the target node. The parameter “N” may represent a configurable or dynamically determined integer value that corresponds to the maximum traversal depth permitted during subnetwork construction.

The system may compute the shortest path length between the target node and each other node using various algorithms, such as BFS, Dijkstra's algorithm, or A path search*, depending on whether or not the edges are weighted. A node is deemed to be within “N” hops if the shortest path length between the node and the target node is less than or equal to “N”. For example, when N=2, only nodes located one or two hops away—i.e., directly connected nodes (Level-1) and their immediate neighbors (Level-2)—are considered within the relevant semantic scope.

In one or more embodiments, the system determines “N” based on query intent or network density. For instance, a user querying for “directly related codes” may trigger N=1, whereas a request for “clinically associated observations” may set N=3. The system may also employ adaptive depth selection, increasing “N” until the marginal gain in semantic relevance falls below a defined threshold or until the number of discovered nodes exceeds a configured limit.

Additionally, the proximity determination may consider edge weights or relationship confidence scores to compute a weighted hop distance rather than a simple edge count. In such embodiments, the effective hop distance between two nodes may be decreased for relationships of high semantic strength (e.g., equivalentTo or broaderThan) but increased for weaker associations (e.g., relatedTo or associatedWith). This allows the system to fine-tune the semantic neighborhood included in the entity-specific network while preserving computational efficiency.

One or more embodiments exclude terms from the entity-specific network (Operation 210). After identifying all nodes within the N-hop proximity, the system applies filtering and exclusion criteria to remove nodes that are irrelevant, redundant, or semantically inconsistent with the target node or user intent. Exclusion criteria may include relationship type filters, terminology scope filters, or contextual relevance thresholds. For example, when constructing a disease-centric network, the system may exclude nodes representing administrative billing codes or non-clinical entities. Similarly, when the query pertains to “lab tests related to diabetes,” the system may filter out nodes corresponding to unrelated medications or procedures.

In one or more embodiments, the system eliminates duplicate or synonymous nodes that represent the same underlying concept across multiple terminologies. For example, if both SNOMED CT and ICD-10 contain nodes equivalent to “Type 2 Diabetes Mellitus,” the system may retain a single canonical node and merge associated relationships into that node to prevent redundancy. This deduplication step may use concept identifiers, normalized text matching, or cross-terminology equivalence mappings maintained in the cross-terminology network.

In one or more embodiments, the system excludes nodes or relationships based on confidence thresholds, versioning metadata, and/or access control policies. For example, the system may omit mappings generated automatically with low confidence and/or repress relationships originating from deprecated terminology versions. Access control policies may additionally restrict inclusion of certain proprietary or unpublished mappings based on user permissions or licensing constraints.

One or more embodiments generate an entity-specific network (also referred to as a specific network) using the terms that have relationships with the target term and that are located within the defined N-hop proximity (Operation 212). The specific network represents a bounded sub-graph of the larger cross-terminology network, including any nodes and edges satisfying the proximity and inclusion criteria.

In one or more embodiments, to generate the specific network, the system instantiates a new graph object or sub-graph view that includes (a) the target node corresponding to the query term, (b) included nodes located within “N” hops of the target node, and (c) edges connecting these nodes, along with their associated relationship metadata (type, direction, confidence, provenance, timestamp).

In one or more embodiments, the system serializes the resulting subnetwork as a machine-interpretable structure, such as an RDF subgraph, a JSON-LD object, or a property graph (e.g., Neo4j format). The system may persist the subnetwork in storage for reuse, cache in memory for immediate query execution, or transmit as part of a response payload to a client interface or API consumer.

In one or more embodiments, the system assigns visual and analytic attributes to the generated network. For example, nodes may be color-coded by terminology source (e.g., SNOMED CT, LOINC, RxNorm), while edge thickness or opacity may represent relationship weight or confidence. Additional graph metrics, such as node centrality, degree distribution, or semantic cohesion score, may be computed and stored as part of the subnetwork metadata to support later analytics or reasoning.

In one or more embodiments, the specific network serves as the input for subsequent query execution, reasoning, or knowledge graph traversal operations. For instance, the system may perform semantic reasoning within the entity-specific network to identify higher-order associations, translate between terminologies, and/or generate query results constrained to a clinically meaningful subgraph.

One or more embodiments dynamically derive an entity-specific network from a large-scale, multi-terminology knowledge graph, focusing on those nodes and relationships that are both semantically relevant and within a defined proximity to the target term. The resulting entity-specific network thus provides a computationally efficient, logically consistent, and domain-focused representation of cross-terminology relationships suitable for subsequent analysis, visualization, or clinical decision support.

One or more embodiments execute the query using the entity-specific network (Operation 214). The query execution process operates on the entity-specific network that represents a bounded subgraph including terms and relationships within a defined number of hops from the target term. The system traverses the entity-specific network beginning at the target node and follows edges that satisfy the query parameters, such as relationship type, directionality, or minimum confidence score.

In one or more embodiments, the query may request specific types of relationships (e.g., equivalentTo, broaderThan, relatedTo), term categories (e.g., medication, diagnosis, lab test), or specific ontology sources (e.g., SNOMED CT, LOINC, RxNorm). The system applies these constraints to the traversal algorithm to ensure that only relevant nodes and edges are explored. For example, a query, such as “show all laboratory tests related to diabetes,” may trigger a traversal limited to relatedTo or associatedWith edges that connect the “Diabetes Mellitus” node to descendant nodes within the “lab test” domain of the network. The system may implement the traversal using a graph search algorithm, such as Depth-First Search (DFS), BFS, or Bidirectional Search, depending on the query's structure and optimization goals. To reduce computational overhead, the system may employ heuristic-based traversal strategies that prioritize edges and nodes with higher semantic weight, such as high-confidence mappings or frequently co-occurring clinical relationships.

In one or more embodiments, the system dynamically manages traversal depth and breadth to optimize performance. The system may limit the traversal by (a) the configured number of hops (N), (b) a maximum number of nodes to explore, (c) a cumulative edge-weight threshold, and/or (d) execution time constraints. This ensures the system balances query completeness against resource efficiency. Additionally, the system may utilize in-memory caching or indexing structures to accelerate access to frequently queried nodes or precomputed relationship clusters. The system may apply path scoring algorithms to rank discovered relationships or sub-paths based on semantic relevance, confidence, or clinical importance. For example, a high-confidence equivalentTo relationship between ICD-10:E11 and SNOMED CT:44054006 may be assigned a higher score than a loosely associated relatedTo path between Diabetes Mellitus and Dietary Counseling. The path scores may be computed using a weighted combination of edge confidence, relationship type, and traversal depth, enabling the system to prioritize more meaningful results during query execution.

In one or more embodiments, executing the query includes aggregating and formatting the retrieved nodes and edges according to the query requirements. Once relevant nodes and relationships are identified, the system compiles the results into a structured output that may be delivered to a user interface, reporting engine, or downstream analytics module. The system may group retrieved entities by terminology type, relationship type, and/or semantic proximity to the target node. For example, results may be organized into various sections, such as “Equivalent Terms,” “Related Laboratory Tests,” and “Associated Medications.” Each section may include node identifiers, preferred labels, terminology source, and/or relationship metadata.

In one or more embodiments, the system formats the results as an interactive knowledge graph visualization, a tabular output, or a JSON response object. Visual representations may employ layout algorithms (e.g., force-directed, radial, or hierarchical) to position nodes according to hop distance or relationship strength. Nodes and edges may be color-coded or weighted to visually convey terminology source, relationship confidence, or semantic type. This enables users to quickly interpret complex inter-terminology relationships and derive actionable insights. The system may compute and display aggregate metrics, such as the number of terms retrieved per terminology, average relationship weight, or most frequent relationship types. These analytics provide an overview of how extensively the target entity is represented or connected across domains, helping users evaluate data completeness and interoperability.

In one or more embodiments, executing the query includes comparing information retrieved from the entity-specific network with patient data. More particularly, the system extracts information from the entity-specific network that corresponds to the target entity, such as related diagnoses, laboratory tests, procedures, or medications, and compares that information against patient-specific data retrieved from one or more clinical data sources (e.g., EHR systems, FHIR APIs, or claims repositories). The comparison process may involve matching concept identifiers, textual labels, and/or vector embeddings between the graph-derived entities and patient-record entities. For example, the system may determine if the patient's EHR includes records corresponding to lab tests linked to Diabetes Mellitus within the entity-specific network, such as “Hemoglobin A1c (LOINC:4548-4).” When overlap is detected, the system may quantify the overlap as a coverage ratio or match score, indicating the degree of alignment between the patient's observed data and the canonical or expected relationships derived from the network. Conversely, when certain nodes from the entity-specific network have no corresponding data entries in the patient record, the system may identify these as missing data elements or clinical gaps.

In one or more embodiments, the system presents missing or mismatched data as actionable recommendations for the healthcare provider. For example, when a patient diagnosed with Type 2 Diabetes Mellitus has no corresponding HbA1c test result within the recommended timeframe, the system may generate an alert suggesting the need for that test. Similarly, when patient data includes superfluous or inconsistent entries, such as redundant diagnoses or incompatible medication combinations, the system may generate a data-quality alert to notify the user of potential errors or contradictions.

In one or more embodiments, the comparison step employs semantic similarity metrics or vector-based matching between network-derived concept embeddings and patient-record embeddings. This allows the system to detect approximate or conceptually related matches even when the patient data uses different terminology systems or local codes. The system may display comparison results in a dashboard interface, highlighting confirmed matches, missing concepts, and/or anomalous data.

By performing query execution and patient-data comparison using the entity-specific network, one or more embodiments provide a technical mechanism for contextualizing patient information against a semantically normalized reference structure. This enables intelligent detection of clinical gaps, supports decision-making, and/or improves the quality and completeness of healthcare data analytics and interoperability workflows.

One or more embodiments present query results to the user (Operation 216). After the system executes the query using the entity-specific network, the resulting data, including nodes, relationships, inferred connections, and/or any patient-specific comparisons, is prepared for presentation through a user interface or application dashboard. The presentation component may transform the raw query results into a human-readable, consumable format, allowing clinicians, data analysts, or system operators to efficiently interpret the findings.

In one or more embodiments, the system presents the query results in one or more output formats. These formats include natural language summaries, structured data tables, graphical visualizations, and/or hybrids views. Natural language summaries are generated by natural-language generation (NLG) components that convert structured data and semantic relationships into explanatory text (e.g., “The patient has a diagnosis of Type 2 Diabetes Mellitus and lacks a recent HbA1c test result.”). Structured data tables include lists of related terms, relationship types, confidence scores, and terminology sources (e.g., SNOMED CT, LOINC, RxNorm). Graphical visualizations include knowledge graphs that display nodes and edges arranged according to semantic proximity or relationship strength. Hybrid views combine textual explanations with structured tables or graph visualizations to deliver both interpretability and analytical depth.

In one or more embodiments, the system uses interactive visualization components that enable the user to explore the network by expanding or collapsing nodes, filtering by terminology, adjusting hop depth, and/or hovering over nodes to reveal metadata (e.g., definition, code, relationship source). Each visualization may include color-coding, edge weighting, and confidence overlays to visually indicate data quality and relationship type.

In one or more embodiments, the system applies context-adaptive presentation logic to tailor the output to the query type, user role, and/or device. For example, a clinician may receive a concise clinical summary and recommended next steps, whereas a data engineer may receive a graph visualization and detailed relationship metadata. Similarly, results may be formatted differently, depending on whether the interface is rendered on a desktop dashboard, a mobile device, or integrated into an EHR interface.

In one or more embodiments, the system provides explanatory context derived from the reasoning process. Explanatory context may include how a specific relationship was inferred, the data sources that contributed to the mapping, and/or the associated confidence level. For example, when showing an inferred link between “Diabetes Mellitus” and “HbA1c Laboratory Test,” the interface may include a note such as “Inferred based on co-occurrence in SNOMED CT and LOINC mappings, confidence 0.93.” This transparency enhances user trust and supports auditability of AI-derived outputs.

In one or more embodiments, the system employs ML techniques to identify and generate a template for presenting the query results to the user. The presentation templates may define layout structure, content ordering, and/or display modality suitable for different types of queries and users. Initially, the system may analyze the entity-specific network and its associated query results to detect semantic patterns, data gaps, and/or clinically relevant findings that may warrant emphasis. For example, the system may identify clusters of strongly related nodes (e.g., diagnosis-lab-medication triads), relationships indicating potential clinical gaps (e.g., missing lab data), and/or anomalies (e.g., inconsistent code mappings). Based on this analysis, the system uses one or more ML models, such as a template-selection classifier or reinforcement learning policy model, to predict the most appropriate presentation template. The model may consider various input features, including query intent, target term type (e.g., disease, test, medication), relationship density, missing data patterns, user role, and display context. The template defines whether the output should be rendered as a textual summary, an analytical dashboard, a hierarchical table, and/or an interactive knowledge graph.

In one or more embodiments, once the template is selected, the system populates the template with relevant data elements, including terms, relationships, reasoning explanations, and, where applicable, patient data or comparison results. The system may map data elements to corresponding presentation slots, e.g., summary section, table column, or graph node. The system may then render the populated template in the user interface. For example, a clinical-summary template may display a top-level narrative followed by actionable recommendations (e.g., “Patient has diabetes; no HbA1c result in past six months. Recommend ordering HbA1c test.”). A terminology-alignment template may display a table of mapped codes across ICD-10, SNOMED CT, and RxNorm, grouped by equivalence or association. A network-exploration template may visualize the subnetwork surrounding the target term with expandable nodes and relationship filters.

In one or more embodiments, the system enables adaptive re-templating based on user feedback or interaction history. For instance, if a user frequently expands graph views instead of reading natural-language summaries, the system may learn to prioritize visual graph templates for that user in future sessions.

In one or more embodiments, automatically identifying and applying an optimal presentation template ensures that query results, derived from complex cross-terminology reasoning, are delivered in an intelligible, context-appropriate, and actionable form. This ML-driven presentation framework allows clinicians and analysts to quickly understand semantic relationships, detect data gaps, and make informed decisions based on the entity-specific network's output.

One or more embodiments perform one or more actions based on the query results (Operation 218). After presenting the query results to the user, the system determines one or more follow-up actions to execute in response to the findings derived from the entity-specific network and/or the comparison with patient data. The system may automatically initiate actions based on detection of a triggering event. Alternatively, actions may require manual initiation by a user via a user interface.

In one or more embodiments, the type of action performed in response to a query results depends on the query context, the type of entity involved (e.g., diagnosis, laboratory test, medication, procedure), and/or the results generated from the cross-terminology reasoning process. The system analyzes the query output to detect actionable conditions, such as missing clinical data, conflicting mappings, or treatment gaps, and associates these conditions with predefined action templates or workflow rules. For example, when the query identifies a missing laboratory test associated with a diagnosed condition, the system may automatically generate a clinical recommendation or task request. When the target term corresponds to “Type 2 Diabetes Mellitus” and the entity-specific network reveals that “HbA1c test (LOINC:4548-4)” is an expected associated entity not found in the patient record, the system may initiate a responsive action. The action may include (a) automatically generating a lab order suggestion for the missing test, (b) sending an alert or notification to the treating provider, and/or (c) updating a clinical dashboard to flag the missing observation as an open quality metric item.

In one or more embodiments, the system integrates with external systems through APIs or secure message exchanges to execute actions beyond the analytics layer. This may include the following: (a) EHR system integration-inserting an alert, recommendation, or order into the patient's chart or workflow queue; (b) care coordination systems-creating or updating tasks for nursing staff or care managers; (c) analytics platforms-logging the identified gaps or findings for population-level tracking and quality reporting; and/or (d) terminology governance modules—updating mapping accuracy feedback to improve cross-terminology consistency for future queries.

In one or more embodiments, actions responsive to query results may include data governance or curation tasks triggered by discrepancies detected in the entity-specific network. For example, if the query results reveal inconsistent or duplicate mappings between SNOMED CT and ICD-10 codes, the system may generate a mapping review task for terminology administrators or initiate an automatic correction in a staging environment, pending validation.

In one or more embodiments, the system applies ML models to predict a most appropriate next action based on prior outcomes, user preferences, and/or contextual metadata. For example, a recommendation model may rank possible actions, such as “notify clinician,” “update mapping,” and/or “add to watchlist,” based on the historical acceptance or rejection of similar suggestions.

In one or more embodiments, performing an action includes updating the cross-terminology knowledge base and/or user-specific profiles to record the outcome of the action. For example, when a user accepts a recommendation or corrects a mapping, the system may store this as a feedback event that influences future reasoning, presentation templates, and/or traversal priorities. This establishes a closed-loop learning system, allowing continuous improvement of both the cross-terminology network and the downstream clinical or analytical workflows.

In one or more embodiments, the system presents a confirmation interface or interactive action panel where the user can review, modify, and/or approve automatically suggested actions before execution. This ensures transparency and user oversight in cases where actions involve clinical decision support, patient treatment, and/or terminology modification.

In one or more embodiments, the system logs executed actions, associated context (query parameters, target entity, reasoning path), and/or user responses for auditability and traceability. The system may store the logs in a secure repository compliant with healthcare data standards (e.g., HIPAA, HL7 FHIR AuditEvent), enabling retrospective review and/or compliance verification.

In one or more embodiments, executing the first query based on the terms associated with nodes in the limited network includes identifying one or more treatment or intervention codes associated with the first term. The limited network may include nodes that represent procedural codes (e.g., CPT, HCPCS) or treatment protocol identifiers (e.g., SNOMED CT procedures, RxNorm medications) connected to diagnostic or finding terms through inter-terminology and intra-terminology edges. For example, when a query is received for a proprietary term “Stage 2 Hypertension” within a provider's local terminology, the limited network generated around that term includes neighboring nodes mapped to standardized treatment codes such as CPT 99214 (Office Visit for Hypertension Management), RxNorm 617314 (Lisinopril 10 mg oral tablet), and SNOMED CT 182832007 (Antihypertensive therapy). Based on graph traversal and edge-weight analysis, the system identifies these codes as corresponding treatment or intervention concepts for the first term.

In one or more embodiments, once the treatment or intervention codes are identified, the computing system initiates one or more treatment actions. The treatment actions can include the following: (a) automatically generating an order set within a computerized provider order entry (CPOE) interface, (b) pre-populating prescription details for clinician confirmation, (c) generating follow-up tasks (e.g., blood pressure monitoring reminders), and/or (d) initiating patient education workflows. In an example, the system automatically generates a draft medication order for Lisinopril 10 mg PO daily and a follow-up appointment request two weeks after initiation. The provider interface presents these auto-generated elements for review, modification, and approval.

In one or more embodiments, executing the first query includes identifying one or more clinical guidelines associated with the first term. The limited network may include nodes representing guideline identifiers (e.g., USPSTF, NCCN, or specialty-society recommendations) connected to relevant diagnostic and procedure codes. For example, when the first term corresponds to “Colorectal Cancer Screening,” the limited network links to guideline nodes that represent the U.S. Preventive Services Task Force (USPSTF) recommendations for colorectal cancer screening. The system retrieves the guideline metadata, including recommended diagnostic procedures (CPT 45378, Colonoscopy) and indication codes (ICD-10 Z12.11, Encounter for screening for malignant neoplasm of colon).

In one or more embodiments, the system then evaluates the patient's demographic and clinical attributes (e.g., age, prior screening history, family history) against guideline criteria. Based on this evaluation, the system determines whether a diagnostic test or treatment procedure is indicated. If indicated, the system automatically schedules a medical procedure in the relevant scheduling subsystem. For example, when a 52-year-old patient with no prior colonoscopy meets inclusion criteria, the system reserves an available endoscopy slot, associates the appointment with CPT 45378, and/or populates pre-procedure instructions and consent forms.

In one or more embodiments, executing the first query includes identifying one or more medication terms within the limited network and determining if a contraindication exists. Each node in the limited network can include clinical relationships, such as “interacts with,” “contraindicated in,” or “adverse effect of,” that connect medication nodes to condition or finding nodes. For example, when the system processes a query associated with a medication term “Warfarin,” the limited network includes an edge to SNOMED CT 443883004 (Pregnancy), representing a contraindication relationship. If the patient's record includes the pregnancy condition node, the system identifies a contraindication.

In one or more embodiments, upon detecting a contraindication, the system generates a safety alert that includes the evidence supporting the contraindication and/or one or more recommended alternative treatments. The safety alert may display: “Contraindicated: Warfarin use during pregnancy,” include the relationship graph showing nodes and edges that produced the alert, and suggest alternative therapies such as Enoxaparin (RxNorm 855332). The safety alert may further include an explainability panel with evidence sentences, confidence scores, and/or provenance information for regulatory auditing. The system may transmit the safety alert to a clinician interface. These operations improve the safety and reliability of electronic prescribing systems by programmatically enforcing contraindication detection derived from the structured semantics of the limited network.

In one or more embodiments, executing the first query further includes determining if prior authorization is required for a procedure or medication identified in the query results. Nodes in the limited network may be enriched with payer-specific attributes that indicate authorization policies, pre-certification thresholds, and/or documentation requirements. For example, when the query result identifies CPT 45378 (Diagnostic Colonoscopy) as a recommended procedure, the system accesses metadata linked to that node indicating that prior authorization is required for patients under certain payer plans. The system retrieves the payer rule through an integrated API or a stored policy mapping. The system may then generate a prior authorization request populated with clinical justification data derived from nodes of the limited network, such as indication codes (ICD-10 Z12.11), abnormal lab findings (LOINC 29771-3: Occult blood positive), and applicable guideline references. The system may compose the request in a standardized electronic format (e.g., X12 278 or FHIR PA Bundle) and may transmits the request to the payer system. Upon receipt of authorization approval, the system may automatically update a scheduling subsystem to confirm the procedure, attach the authorization number to the patient encounter, and/or record the transaction in an audit log.

### 4. Example Embodiment

A detailed example is described below for purposes of clarity. Components and/or operations described below should be understood as one specific example that may not be applicable to certain embodiments. Accordingly, components and/or operations described below should not be construed as limiting the scope of any of the claims.

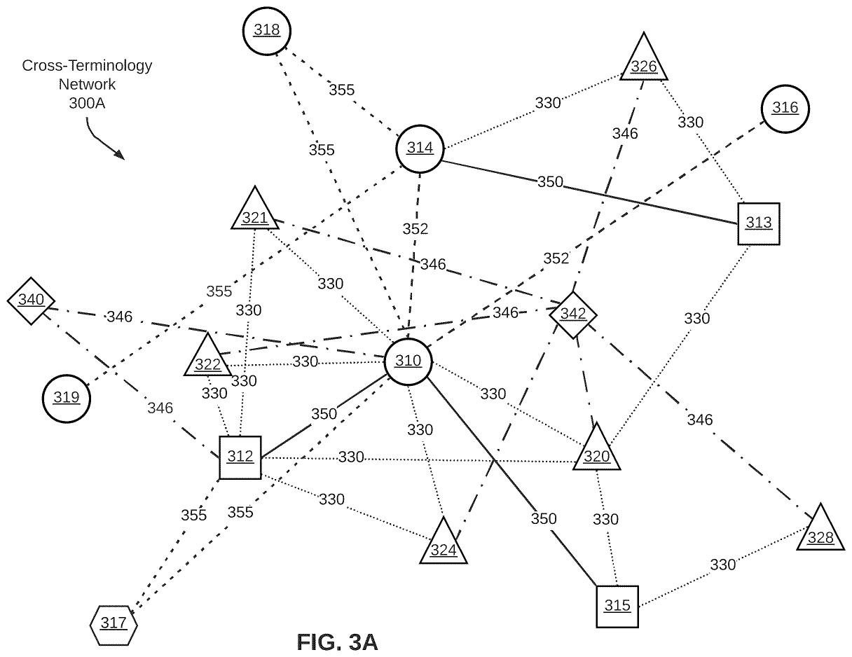

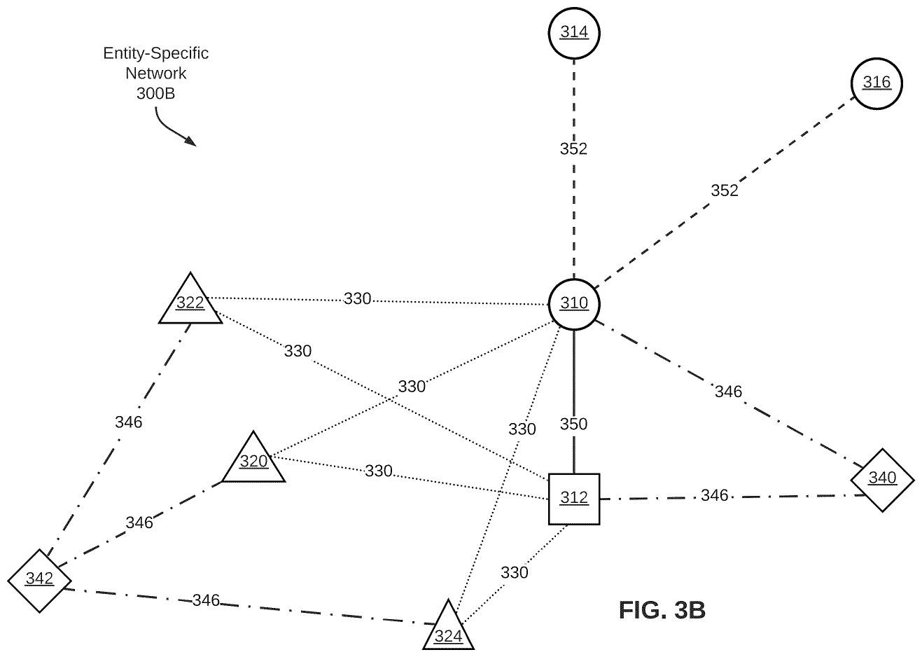

FIG. 3A illustrates an example embodiment of a cross-domain or cross-terminology network. Cross-terminology network 300A models a domain-level graph unifying standardized and proprietary medical code systems. Cross-domain network 300A is a semantic superset that encodes relationships among terminology entities, ontology concepts, contextual attributes, and provenance metadata. The architecture allows a mapping engine to traverse, validate, and extract query-specific networks such as entity-specific network 300B shown in FIG. 3B.

Nodes in cross-terminology network 300A belong to defined node types identifying functional classes. Node types include the following: (a) a standard code type, representing globally standardized codes (e.g., LOINC, SNOMED CT, RxNorm, ICD); (b) a proprietary code type, representing local or vendor-specific identifiers (e.g., Cerner Code Set 72, internal EHR codes, LIS identifiers); (c) an attribute type, representing contextual properties, such as specimen, property, or unit; (d) a provenance type, representing models, rules, or lineage entities; and (e) a reference-concept type, representing ontology anchors linking multiple terminologies.

Edges in cross-terminology network 300A define relationships types between the node types (e.g., proprietary→standard for mappings, code→attribute for context, model→code for provenance).

Cross-terminology network 300A includes standard code nodes that correspond to globally standardized laboratory test concepts. Standard code nodes include a first standard code node 310 corresponding to Hemoglobin (HGB) Test (LOINC), a second standard code node 314 corresponding to Hematocrit (HCT) Test (LOINC), a third standard code node 316 corresponding to Glucose Test (LOINC), a fourth standard code node 318 corresponding to complete blood count (CBC) Panel (LOINC), and a fifth standard code node 319 corresponding to red blood cell (RBC) Count (LOINC). The standard code nodes store a persistent identifier (e.g., LOINC ID), preferred display name, and metadata, such as property, specimen, and unit definitions. Standard code nodes form a canonical reference layer of cross-terminology network and act as stable mapping targets.

Cross-terminology network 300A includes proprietary code nodes that correspond to local vendor identifiers. Proprietary code nodes include a first proprietary code node 312 corresponding to HGB Observation, a second proprietary code node 313 corresponding to HCT Observation, and a third proprietary code node 315 corresponding to HGB Result. The first proprietary code node 312 (HGB Observation) and the second proprietary code node 313 (HCT Observation) are from Cerner Code Set 72. The third proprietary code node 315 is from another vendor's laboratory information system (LIS), e.g., Epic Beaker, Meditech LIS, or Sunquest Lab. The proprietary code nodes may encode additional local context, naming conventions, or measurement methodologies. Each proprietary code node includes attributes describing its local schema, format, or implementation context.

Cross-terminology network 300A includes mapping edges (solid lines). The mapping edges connect standard code nodes and proprietary code nodes. The mapping edges represent validated or high-confidence equivalences. The mapping edges are generated by applying an embedding-based similarity model that computes vector representations for each code based on textual descriptions, synonyms, and attribute metadata. A model node 340 stores the parameters of the embedding model (ClinicalEmbedding-v1), and a rule node 342 stores the logical rule set (AttributeGuard-v2) used for post-similarity validation. Each mapping edge may include an associated similarity score and provenance reference to model node 340 and rule node 342.

A reference-concept node 317 serves as an ontology anchor (e.g., SNOMED CT Hemoglobin Measurement) connected to both standard and proprietary code nodes via alignment edges. Alignment edges unify heterogeneous code systems by linking the nodes to shared upper-ontology concepts, enabling cross-terminology inference and multi-system interoperability.

Cross-terminology network 300A includes a plurality of attribute nodes. Attribute nodes define contextual equivalence guards that constrain valid mappings. These include the following: a first attribute node 320 corresponding to Specimen: Blood (Venous); a second attribute node 321 corresponding to Specimen: Blood (Capillary); a third attribute node 322 corresponding to Property: Mass/Volume; a fourth attribute node 324 corresponding to Units: g/dL; a fifth attribute node 326 corresponding to Property: Volume Fraction; and a sixth attribute node 328 corresponding to Units: g/L. Attribute edges (dotted lines) associate code nodes with their contextual attributes, ensuring that a mapping is only accepted when both source and target share compatible clinical characteristics.

In cross-terminology network 300A, a provenance layer provides lineage for all generated relationships. Model node 340 represents the trained embedding model, storing version, training corpus, and hyperparameters (e.g., vector dimension=768, learning rate=1e-5). Rule node 342 defines the deterministic post-processing rules, e.g., enforcing that mapped codes share identical specimen and property attributes. Provenance edges 346 link model node 340 and rule node 342 to governed entities, providing a traceable audit path that supports explainability and compliance.

Cross-terminology network 300A includes additional edge types that enrich the topology. The additional edge types include similarity edges 352, membership edges 355, attribute edges 330, and provenance edges 346. Similarity edges 352 connect related but non-identical nodes, e.g., first standard code node 310 (HGB Test)↔second standard code node 314 (HCT Test) or first standard code node 310 (HGB Test)↔third standard code node 316 (Glucose Test). Similarity edges 352 represent semantic proximity for clustering or query expansion. Membership edges 355 link component tests (e.g., first standard code node 310 (HGB Test), second standard code node 314 (HCT Test), fifth standard code node 319 (RBC Count) to fourth standard code node 318 (CBC Panels). Attribute edges 330 link standard code nodes and proprietary code nodes to attribute nodes. Provenance edges 346 link proprietary code nodes, standard code nodes, and attribute nodes to model node 340 and rule node 342.

By explicitly modeling node types and edge semantics, cross-terminology network 300A supports structured reasoning. A query processor may, for instance, restrict traversal to maps_to edges between proprietary code and standard code types or may filter paths where attributes differ. The result is a graph architecture that enables explainable, reproducible, and machine-validated terminology alignment across diverse healthcare systems.

FIG. 3B illustrates entity-specific network 300B. Entity-specific network 300B is a dynamically extracted subgraph derived from the cross-terminology network 300A (FIG. 3A). The limited network represents the subset of nodes and edges relevant to a specific mapping or query generated by applying a hop-limited traversal operation that bounds graph expansion to a predefined number of edge transitions from a selected root node.

As illustrated, the system receives a query referencing first standard code node 310 (HGB Test). The subnetwork extraction process identifies the corresponding node as a root and performs a two-hop traversal through the cross-terminology network 300A to collect reachable entities within a maximum path length of two. Each hop represents a single edge transition, such as from a standard code node to a proprietary code node or from a standard code node or proprietary code node to an attribute or provenance node.

Entity-specific network 300B is a two-hop, query-specific network derived from cross-terminology network 300A, retaining the standard-code type, proprietary-code type, attribute type, and provenance type nodes relevant to the mapping of HGB Test and HGB Observation. The hop-limited traversal and pruning yield a contextually rich yet computationally lightweight representation suitable for code-level reasoning, visualization, and clinical interoperability workflows.

At hop 1, the traversal captures directly connected nodes to first standard code node 310 (HGB Test), including the following: first proprietary code node 312 (HGB Observation) via mapping edge 350; second standard code node 314 (HCT Test) and third standard code node 316 (Glucose) via similarity edges 352; and associated first attribute node 320 (Specimen: Blood (Venous)), third attribute node 322 (Property: Mass/Volume), and fourth attribute node 324 (Units: g/dL) via attribute edges 330. Provenance nodes, i.e., model node 340 (ClinicalEmbedding-v1) and rule node 342 (AttributeGuard-v2) are also reached within hop 1 through provenance edges 346.

At hop 2, the traversal includes entities one additional step away from the root, such as secondary attribute relationships connected to first proprietary code node 312 (HGB Observation) or inherited provenance links governing those attributes. Entities beyond two hops, such as unrelated tests, other panels, or higher-level ontology anchors, are excluded to maintain locality and interpretability.

Entity-specific network 300B includes a compact subgraph that includes all entities within two hops of the root node, i.e., first standard code node 310 (HGB Test). This design constrains complexity while preserving sufficient semantic and contextual information for reasoning, visualization, and mapping validation. Each edge in the limited network retains its typed semantics and reference number.

The system may automatically adjust the hop limit parameter (e.g., 1, 2, 3) based on the use case. A one-hop subnetwork yields only direct mappings for high-speed validation. A two-hop subnetwork (as illustrated) preserves both immediate mappings and contextual dependencies. A three-hop subnetwork may be used for ontology-aware reasoning across related tests or panels. By defining hop-bounded extraction, the architecture provides deterministic scalability and interpretability. Traversal cost grows linearly with the number of hops, allowing real-time subnetwork generation even for large ontology graphs. Provenance and attribute edges remain within the same hop-constraint, ensuring that lineage and validation logic are preserved alongside the mapping.

### 5. Machine Learning Architecture

FIG. 4A illustrates a machine learning engine 400 in accordance with one or more embodiments. As illustrated in FIG. 4A, machine learning engine 400 includes input/output module 402, data preprocessing module 404, model selection module 406, training module 408, evaluation and tuning module 410, and inference module 412.

In accordance with an embodiment, input/output module 402 serves as the primary interface for data entering and exiting the system, managing the flow and integrity of data. This module may accommodate a wide range of data sources and formats to facilitate integration and communication within the machine learning architecture.

In an embodiment, an input handler within input/output module 402 includes a data ingestion framework capable of interfacing with various data sources, such as databases, APIs, file systems, and real-time data streams. This framework is equipped with functionalities to handle different data formats (e.g., CSV, JSON, XML) and efficiently manage large volumes of data. It includes mechanisms for batch and real-time data processing that enable the input/output module 402 to be versatile in different operational contexts, whether processing historical datasets or streaming data.

In accordance with an embodiment, input/output module 402 manages data integrity and quality as it enters the system by incorporating initial checks and validations. These checks and validations ensure that incoming data meets predefined quality standards, like checking for missing values, ensuring consistency in data formats, and verifying data ranges and types. This proactive approach to data quality minimizes potential errors and inconsistencies in later stages of the machine learning process.

In an embodiment, an output handler within input/output module 402 includes an output framework designed to handle the distribution and exportation of outputs, predictions, or insights. Using the output framework, input/output module 402 formats these outputs into user-friendly and accessible formats, such as reports, visualizations, or data files compatible with other systems. Input/output module 402 also ensures secure and efficient transmission of these outputs to end-users or other systems in an embodiment and may employ encryption and secure data transfer protocols to maintain data confidentiality.

In accordance with an embodiment, data preprocessing module 404 transforms data into a format suitable for use by other modules in machine learning engine 400. For example, data preprocessing module 404 may transform raw data into a normalized or standardized format suitable for training ML models and for processing new data inputs for inference. In an embodiment, data preprocessing module 404 acts as a bridge between the raw data sources and the analytical capabilities of machine learning engine 400.

In an embodiment, data preprocessing module 404 begins by implementing a series of preprocessing steps to clean, normalize, and/or standardize the data. This involves handling a variety of anomalies, such as managing unexpected data elements, recognizing inconsistencies, or dealing with missing values. Some of these anomalies can be addressed through methods like imputation or removal of incomplete records, depending on the nature and volume of the missing data. Data preprocessing module 404 may be configured to handle anomalies in different ways depending on context. Data preprocessing module 404 also handles the normalization of numerical data in preparation for use with models sensitive to the scale of the data, like neural networks and distance-based algorithms. Normalization techniques, such as min-max scaling or z-score standardization, may be applied to bring numerical features to a common scale, enhancing the model's ability to learn effectively.

In an embodiment, data preprocessing module 404 includes a feature encoding framework that ensures categorical variables are transformed into a format that can be easily interpreted by machine learning algorithms. Techniques like one-hot encoding or label encoding may be employed to convert categorical data into numerical values, making them suitable for analysis. The module may also include feature selection mechanisms, where redundant or irrelevant features are identified and removed, thereby increasing the efficiency and performance of the model.

In accordance with an embodiment, when data preprocessing module 404 processes new data for inference, data preprocessing module 404 replicates the same preprocessing steps to ensure consistency with the training data format. This helps to avoid discrepancies between the training data format and the inference data format, thereby reducing the likelihood of inaccurate or invalid model predictions.

In an embodiment, model selection module 406 includes logic for determining the most suitable algorithm or model architecture for a given dataset and problem. This module operates in part by analyzing the characteristics of the input data, such as its dimensionality, distribution, and the type of problem (classification, regression, clustering, etc.).

In an embodiment, model selection module 406 employs a variety of statistical and analytical techniques to understand data patterns, identify potential correlations, and assess the complexity of the task. Based on this analysis, it then matches the data characteristics with the strengths and weaknesses of various available models. This can range from simple linear models for less complex problems to sophisticated deep learning architectures for tasks requiring feature extraction and high-level pattern recognition, such as image and speech recognition.

In an embodiment, model selection module 406 utilizes techniques from the field of Automated Machine Learning (AutoML). AutoML systems automate the process of model selection by rapidly prototyping and evaluating multiple models. They use techniques like Bayesian optimization, genetic algorithms, or reinforcement learning to explore the model space efficiently. Model selection module 406 may use these techniques to evaluate each candidate model based on performance metrics relevant to the task. For example, accuracy, precision, recall, or F1 score may be used for classification tasks and mean squared error metrics may be used for regression tasks. Accuracy measures the proportion of correct predictions (both positive and negative). Precision measures the proportion of actual positives among the predicted positive cases. Recall (also known as sensitivity) evaluates how well the model identifies actual positives. F1 Score is a single metric that accounts for both false positives and false negatives. The mean squared error (MSE) metric may be used for regression tasks. MSE measures the average squared difference between the actual and predicted values, providing an indication of the model's accuracy. A lower MSE may indicate a model's greater accuracy in predicting values, as it represents a smaller average discrepancy between the actual and predicted values.

In accordance with an embodiment, model selection module 406 also considers computational efficiency and resource constraints. This is meant to help ensure the selected model is both accurate and practical in terms of computational and time requirements. In an embodiment, certain features of model selection module 406 are configurable such as a configured bias toward (or against) computational efficiency.

In accordance with an embodiment, training module 408 manages the ‘learning’ process of ML models by implementing various learning algorithms that enable models to identify patterns and make predictions or decisions based on input data. In an embodiment, the training process begins with the preparation of the dataset after preprocessing; this involves splitting the data into training and validation sets. The training set is used to teach the model, while the validation set is used to evaluate its performance and adjust parameters accordingly. Training module 408 handles the iterative process of feeding the training data into the model, adjusting the model's internal parameters (like weights in neural networks) through backpropagation and optimization algorithms, such as stochastic gradient descent or other algorithms providing similarly useful results.

In accordance with an embodiment, training module 408 manages overfitting, where a model learns the training data too well, including its noise and outliers, at the expense of its ability to generalize to new data. Techniques such as regularization, dropout (in neural networks), and early stopping are implemented to mitigate this. Additionally, the module employs various techniques for hyperparameter tuning; this involves adjusting model parameters that are not directly learned from the training process, such as learning rate, the number of layers in a neural network, or the number of trees in a random forest.

In an embodiment, training module 408 includes logic to handle different types of data and learning tasks. For instance, it includes different training routines for supervised learning (where the training data comes with labels) and unsupervised learning (without labeled data). In the case of deep learning models, training module 408 also manages the complexities of training neural networks that include initializing network weights, choosing activation functions, and setting up neural network layers.

In an embodiment, evaluation and tuning module 410 incorporates dynamic feedback mechanisms and facilitates continuous model evolution to help ensure the system's relevance and accuracy as the data landscape changes. Evaluation and tuning module 410 conducts a detailed evaluation of a model's performance. This process involves using statistical methods and a variety of performance metrics to analyze the model's predictions against a validation dataset. The validation dataset, distinct from the training set, is instrumental in assessing the model's predictive accuracy and its capacity to generalize beyond the training data. The module's algorithms meticulously dissect the model's output, uncovering biases, variances, and the overall effectiveness of the model in capturing the underlying patterns of the data.

In an embodiment, evaluation and tuning module 410 performs continuous model tuning by using hyperparameter optimization. Evaluation and tuning module 410 performs an exploration of the hyperparameter space using algorithms, such as grid search, random search, or more sophisticated methods like Bayesian optimization. Evaluation and tuning module 410 uses these algorithms to iteratively adjust and refine the model's hyperparameters—settings that govern the model's learning process but are not directly learned from the data—to enhance the model's performance. This tuning process helps to balance the model's complexity with its ability to generalize and attempts to avoid the pitfalls of underfitting or overfitting.

In an embodiment, evaluation and tuning module 410 integrates data feedback and updates the model. Evaluation and tuning module 410 actively collects feedback from the model's real-world applications, an indicator of the model's performance in practical scenarios. Such feedback can come from various sources depending on the nature of the application. For example, in a user-centric application like a recommendation system, feedback might comprise user interactions, preferences, and responses. In other contexts, such as predicting events, it might involve analyzing the model's prediction errors, misclassifications, or other performance metrics in live environments.

In an embodiment, feedback integration logic within evaluation and tuning module 410 integrates this feedback using a process of assimilating new data patterns, user interactions, and error trends into the system's knowledge base. The feedback integration logic uses this information to identify shifts in data trends or emergent patterns that were not present or inadequately represented in the original training dataset. Based on this analysis, the module triggers a retraining or updating cycle for the model. If the feedback suggests minor deviations or incremental changes in data patterns, the feedback integration logic may employ incremental learning strategies, fine-tuning the model with the new data while retaining its previously learned knowledge. In cases where the feedback indicates significant shifts or the emergence of new patterns, a more comprehensive model updating process may be initiated. This process might involve revisiting the model selection process, re-evaluating the suitability of the current model architecture, and/or potentially exploring alternative models or configurations that are more attuned to the new data.

In accordance with an embodiment, throughout this iterative process of feedback integration and model updating, evaluation and tuning module 410 employs version control mechanisms to track changes, modifications, and the evolution of the model, facilitating transparency and allowing for rollback if necessary. This continuous learning and adaptation cycle, driven by real-world data and feedback, helps to endure the model's ongoing effectiveness, relevance, and accuracy.

In an embodiment, inference module 412 transforms data raw data into actionable, precise, and contextually relevant predictions. In addition to processing and applying a trained model to new data, inference module 412 may also include post-processing logic that refines the raw outputs of the model into meaningful insights.

In an embodiment, inference module 412 includes classification logic that takes the probabilistic outputs of the model and converts them into definitive class labels. This process involves an analytical interpretation of the probability distribution for each class. For example, in binary classification, the classification logic may identify the class with a probability above a certain threshold, but classification logic may also consider the relative probability distribution between classes to create a more nuanced and accurate classification.

In an embodiment, inference module 412 transforms the outputs of a trained model into definitive classifications. Inference module 412 employs the underlying model as a tool to generate probabilistic outputs for each potential class. It then engages in an interpretative process to convert these probabilities into concrete class labels.

In an embodiment, when inference module 412 receives the probabilistic outputs from the model, it analyzes these probabilities to determine how they are distributed across some or every potential class. If the highest probability is not significantly greater than the others, inference module 412 may determine that there is ambiguity or interpret this as a lack of confidence displayed by the model.

In an embodiment, inference module 412 uses thresholding techniques for applications where making a definitive decision based on the highest probability might not suffice due to the critical nature of the decision. In such cases, inference module 412 assesses if the highest probability surpasses a certain confidence threshold that is predetermined based on the specific requirements of the application. If the probabilities do not meet this threshold, inference module 412 may flag the result as uncertain or defer the decision to a human expert. Inference module 412 dynamically adjusts the decision thresholds based on the sensitivity and specificity requirements of the application, subject to calibration for balancing the trade-offs between false positives and false negatives.

In accordance with an embodiment, inference module 412 contextualizes the probability distribution against the backdrop of the specific application. This involves a comparative analysis, especially in instances where multiple classes have similar probability scores, to deduce the most plausible classification. In an embodiment, inference module 412 may incorporate additional decision-making rules or contextual information to guide this analysis, ensuring that the classification aligns with the practical and contextual nuances of the application.

In regression models, where the outputs are continuous values, inference module 412 may engage in a detailed scaling process in an embodiment. Outputs, often normalized or standardized during training for optimal model performance, are rescaled back to their original range. This rescaling involves recalibration of the output values using the original data's statistical parameters, such as mean and standard deviation, ensuring that the predictions are meaningful and comparable to the real-world scales they represent.

In an embodiment, inference module 412 incorporates domain-specific adjustments into its post-processing routine. This involves tailoring the model's output to align with specific industry knowledge or contextual information. For example, in financial forecasting, inference module 412 may adjust predictions based on current market trends, economic indicators, or recent significant events, ensuring that the outputs are both statistically accurate and practically relevant.

In an embodiment, inference module 412 includes logic to handle uncertainty and ambiguity in the model's predictions. In cases where inference module 412 outputs a measure of uncertainty, such as in Bayesian inference models, inference module 412 interprets these uncertainty measures by converting probabilistic distributions or confidence intervals into a format that can be easily understood and acted upon. This provides users with both a prediction and an insight into the confidence level of that prediction. In an embodiment, inference module 412 includes mechanisms for involving human oversight or integrating the instance into a feedback loop for subsequent analysis and model refinement.

In an embodiment, inference module 412 formats the final predictions for end-user consumption. Predictions are converted into visualizations, user-friendly reports, or interactive interfaces. In some systems, like recommendation engines, inference module 412 also integrates feedback mechanisms, where user responses to the predictions are used to continually refine and improve the model, creating a dynamic, self-improving system.

### 6. Machine Learning Engine Operations

FIG. 4B illustrates the operation of a machine learning engine in one or more embodiments. In an embodiment, input/output module 402 receives a dataset intended for training (Operation 401). This data can originate from diverse sources, like databases or real-time data streams, and in varied formats, such as CSV, JSON, or XML. Input/output module 402 assesses and validates the data, ensuring its integrity by checking for consistency, data ranges, and types.

In an embodiment, training data is passed to data preprocessing module 404. Here, the data undergoes a series of transformations to standardize and clean it, making it suitable for training ML models (Operation 403). This involves normalizing numerical data, encoding categorical variables, and handling missing values through techniques like imputation.

In an embodiment, prepared data from the data preprocessing module 404 is then fed into model selection module 406 (Operation 405). This module analyzes the characteristics of the processed data, such as dimensionality and distribution, and selects the most appropriate model architecture for the given dataset and problem. It employs statistical and analytical techniques to match the data with an optimal model, ranging from simpler models for less complex tasks to more advanced architectures for intricate tasks.

In an embodiment, training module 408 trains the selected model with the prepared dataset (Operation 407). It implements learning algorithms to adjust the model's internal parameters, optimizing them to identify patterns and relationships in the training data. Training module 408 also addresses the challenge of overfitting by implementing techniques, like regularization and early stopping, ensuring the model's generalizability.

In an embodiment, evaluation and tuning module 410 evaluates the trained model's performance using the validation dataset (Operation 409). Evaluation and tuning module 410 applies various metrics to assess predictive accuracy and generalization capabilities. It then tunes the model by adjusting hyperparameters, and if needed, incorporates feedback from the model's initial deployments, retraining the model with new data patterns identified from the feedback.

In an embodiment, input/output module 402 receives a dataset intended for inference. Input/output module 402 assesses and validates the data (Operation 411).

In an embodiment, data preprocessing module 404 receives the validated dataset intended for inference (Operation 413). Data preprocessing module 404 ensures that the data format used in training is replicated for the new inference data, maintaining consistency and accuracy for the model's predictions.

In an embodiment, inference module 412 processes the new data set intended for inference, using the trained and tuned model (Operation 415). It applies the model to this data, generating raw probabilistic outputs for predictions. Inference module 412 then executes a series of post-processing steps on these outputs, such as converting probabilities to class labels in classification tasks or rescaling values in regression tasks. It contextualizes the outputs as per the application's requirements, handling any uncertainty in predictions and formatting the final outputs for end-user consumption or integration into larger systems.

In an embodiment, machine learning engine API 414 allows for applications to leverage machine learning engine 400. In an embodiment, machine learning engine API 414 may be built on a RESTful architecture and offer stateless interactions over standard HTTP/HTTPS protocols. Machine learning engine API 414 may feature a variety of endpoints, each tailored to a specific function within machine learning engine 400. In an embodiment, endpoints such as /submitData facilitate the submission of new data for processing, while /retrieveResults is designed for fetching the outcomes of data analysis or model predictions. The MLE API may also include endpoints like /updateModel for model modifications and /trainModel to initiate training with new datasets.

In an embodiment, machine learning engine API 414 is equipped to support SOAP-based interactions. This extension involves defining a WSDL (Web Services Description Language) document that outlines the API's operations and the structure of request and response messages. In an embodiment, machine learning engine API 414 supports various data formats and communication styles. In an embodiment, machine learning engine API 414 endpoints may handle requests in JSON format or any other suitable format. For example, machine learning engine API 414 may process XML, and it may also be engineered to handle more compact and efficient data formats, such as Protocol Buffers or Avro, for use in bandwidth-limited scenarios.

In an embodiment, machine learning engine API 414 is designed to integrate WebSocket technology for applications necessitating real-time data processing and immediate feedback. This integration enables a continuous, bi-directional communication channel for a dynamic and interactive data exchange between the application and machine learning engine 400.

### 7. Generative AI Models

A generative AI model is a machine learning model that is capable of generating new data instances based on the data used to train the model. A generative model may be referred to as a “generative AI model.” Generative models learn the underlying distribution of the training data, enabling them to produce new instances of data that share properties with the original dataset. This capability makes them particularly useful in a variety of applications, including image and voice generation, text synthesis, and more sophisticated tasks like unsupervised learning, semi-supervised learning, and domain adaptation.

One type of generative model is a large language model. Large language models are designed to understand, generate, and interpret human language by processing extensive collections of data. The foundational architecture behind large language models is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times.

In an embodiment, a mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

In accordance with one or more embodiments, transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a SoftMax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

In accordance with one or more embodiments, following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

In accordance with one or more embodiments, integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

In accordance with one or more embodiments, input/output module 402, when used for large language models, handles textual data, converting input text into a format that the model can process. This typically involves tokenization, where the text is broken down into manageable pieces, such as words or sub words, and then converted into numerical representations. These representations, or embeddings, capture semantic information about the text that is then fed into the model for processing. The output from the model is converted from numerical form back into human-readable text, following the generation of predictions or responses.

In accordance with one or more embodiments, data preprocessing module 404 in the context of large language models may include steps such as normalization, where the text is converted to a uniform case and punctuation is standardized. This process ensures that the model treats similar words or symbols consistently, reducing the complexity of the input space. Additionally, techniques such as sentence segmentation may be applied to manage longer texts, enabling the model to process information in chunks that align with natural language structures.

In accordance with one or more embodiments, model selection module 406, when used for large language models involves choosing a specific architecture and configuration that is best suited to the task at hand. This decision is based on various factors, such as the size of the available training data, the complexity of the language tasks to be performed, and computational resource constraints. Models may vary in size from millions to billions of parameters, with larger models generally capable of more nuanced language understanding and generation but requiring significantly more computational power to train and operate.

In accordance with one or more embodiments, training module 408, when used for large language models, is configured to adjust the model's parameters through exposure to training data. This process utilizes optimization algorithms, such as stochastic gradient descent, to minimize the difference between the model's predictions and the actual desired outputs. The training process is computationally intensive, often requiring specialized hardware such as GPUs (Graphics Processing Units) or TPUs (Tensor Processing Units) to manage the large volumes of data and the complexity of the model calculations. During training, techniques, such as dropout and layer normalization, are used to improve model generalization and prevent overfitting (i.e., when a model learns the detail and noise in the training data to the extent that it negatively impacts the model's performance on new data).

In accordance with one or more embodiments, evaluation and tuning module 410 assesses the performance of large language models using metrics such as perplexity, accuracy, and F1 score, depending on the specific language tasks. Evaluation may involve comparing the model's output against a set of labeled validation data, providing insight into how well the model has learned to perform tasks, such as text classification, question answering, or text generation. Tuning involves adjusting model parameters or training strategies based on evaluation outcomes to improve performance. This may include hyperparameter tuning, where parameters that govern the training process, such as learning rate or batch size, are adjusted.

In accordance with one or more embodiments, inference module 412, in the context of large language models, is responsible for generating predictions or responses based on new, unseen data. This process involves feeding the input data through the trained model to produce an output. Inference can be used for a variety of applications, including translating text, generating human-like responses in a chatbot, or summarizing articles.

Another type of generative model is a large multimodal model (LMM). A large multimodal model is an advanced machine learning model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

The self-attention mechanism, which is part of a transformer network, enables the model to weigh the importance of different elements within an input sequence, regardless of their position. This allows the model to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing is a step that ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in machine learning by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides large language models and large multimodal models belong to the broad category of generative models. For example, stochastic models directly incorporate randomness into their structure, making them inherently generative as they can produce a diverse set of outputs for a given input. Generative Adversarial Networks (GANs) learn to generate new data that is indistinguishable from the data they were trained on, using a dual-network architecture that involves a generative component. Variational Autoencoders (VAEs) are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond large language models.

Although generative models can be leveraged for classification tasks, they inherently operate on principles of randomness, leading to a spectrum of possible outcomes in response to identical inputs. Unlike deterministic models that yield a consistent result whenever the same input is given, generative models use the randomness in the data they are trained on to both mimic and diversify from the training data. This diversity makes generative models ideal for generating new and varied data points as well as for tasks that require creativity and novelty. However, a reliance on randomness creates a trade-off between predictability and flexibility for generative models, potentially making them less predictable in scenarios where uniform outcomes may be expected such as classification tasks.

### 8. Practical Applications, Advantages, and Improvements

Embodiments provide several practical applications, advantages, and improvements over existing systems for managing and querying medical terminologies and heterogeneous clinical data. These advantages and improvements include enhanced computational efficiency in semantic reasoning, improved accuracy in terminology alignment and entity mapping, and the ability to automatically generate actionable clinical insights that directly improve patient care and healthcare data quality.

Embodiments improve the accuracy of terminology alignment and query results by using a cross-terminology network that captures both intra-and inter-terminology relationships, including inferred relationships validated by a reasoning engine. For example, when a user queries “diabetes-related lab tests,” the system traverses a semantically consistent graph connecting SNOMED CT, LOINC, and RxNorm entities. This enables the system to identify relevant tests, such as HbA1c (LOINC 4548-4), even if the query term originated in a different terminology or as free text. By grounding retrieved entity in a normalized, ontology-based structure, the system reduces false mappings and increases precision in data retrieval and decision support outputs.

Embodiments improve processing efficiency by dynamically extracting an entity-specific network that includes only nodes and relationships within “N” hops of the target entity. This localized traversal minimizes memory usage and computation time compared to systems that operate over entire ontologies or terminologies. As a result, the system scales efficiently across millions of medical concepts while maintaining low-latency query response, thereby enabling real-time integration into clinical dashboards and analytics pipelines.

Embodiments enhance data completeness by comparing entities in the specific network with patient data to identify missing, redundant, or inconsistent information. For instance, if a patient is diagnosed with Type 2 Diabetes Mellitus but lacks an HbA1c result within the expected period, the system automatically detects this clinical gap and generates a recommendation or alert to order the missing test. This allows healthcare providers to proactively address gaps in care, thereby improving quality-of-care metrics and outcomes.

Embodiments improve upon existing approaches by reducing terminology misalignment errors and manual curation time through automated reasoning and multi-hop traversal over the cross-terminology network.

Embodiments provide a technical benefit by enabling semantically consistent, graph-based query execution across disparate code systems (e.g., SNOMED CT, LOINC, RxNorm) and an economic benefit by reducing the cost and time associated with maintaining manual crosswalk tables and custom ETL mappings.

Embodiments improve the functioning of a computer system by restructuring how healthcare ontologies are queried, i.e., transforming unstructured user queries into machine-readable graph traversals over a dynamically generated subnetwork. This practical application reduces query latency, minimizes unnecessary data processing, and/or yields structured, explainable results that improve interoperability between healthcare systems.

Embodiments implementing automated detection of missing clinical data enable real-time recommendations for laboratory testing, medication adjustments, and/or diagnostic evaluation. This improvement provides a direct clinical benefit by closing care gaps and reducing preventable errors while also improving compliance with quality reporting frameworks (e.g., HEDIS, ONC, CMS MIPS).

By providing automated comparison and feedback mechanisms, embodiments enhance terminology governance. The system identifies inconsistent or obsolete mappings and suggests corrections, ensuring semantic alignment across evolving terminologies.

By providing a unified, machine-interpretable model that extracts an entity-specific network from large-scale, heterogeneous terminologies, embodiments solve a problem rooted in computer technology, namely, the inability of conventional relational or keyword-based systems to perform efficient, context-aware, and/or semantically precise retrieval across diverse coding standards. This improvement results in better system performance, improved user trust, and/or actionable clinical outcomes. The approach improves the functioning of computing systems used in healthcare interoperability and advances the broader technical field of graph-based semantic query execution and ontology reasoning.

The data input to any ML model and/or the data output from any ML model, as described herein, may be used for operations performed by one or more of the following types of software: Database, Cloud Infrastructure, Customer Relationship Management, Data Science, Digital Assistant, Vision, Language, Speech, Forecasting, Enterprise, Middleware, Server, Identity Management, Application Development, Analytics, Security, Data Integration, Health, Hospitality, Retail, Utilities, Operating Systems, Virtualization, Governance and Administration, Migration & Disaster Recovery, Networking, Connectivity, Monitoring, Procurement, Project Management, Risk Management, Supply Chain Management, Manufacturing, Human Capital Management, Customer Experience, Advertising, and Industry-Specific Application.

### 9. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

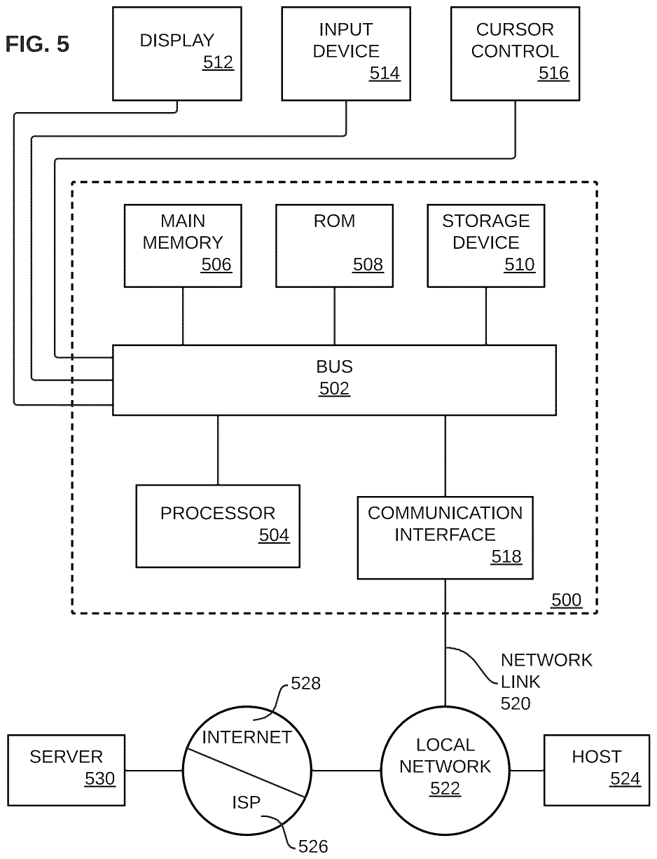

For example, FIG. 5 is a block diagram that illustrates a computer system 500 upon which an embodiment of the disclosure may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read-only memory (ROM) 508 or other static storage device coupled to bus 502 for storing static information and instructions for processor 504. A storage device 510, such as a magnetic disk, optical disk, or a solid-state drive (SSD) is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, SSD, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or SSD of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

### 10. Miscellaneous: Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected, and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, a computer program product includes instructions that, when executed by one or more hardware processors, causes performance of any of the operations described herein and/or recited in any of the claims.

In an embodiment, one or more non-transitory computer-readable storage media store instructions that, when executed by one or more hardware processors, cause performance of any of the operations described herein and/or recited in any of the claims. As used herein, the term “non-transitory computer-readable medium” refers to any tangible storage medium that stores computer-executable instructions for execution by one or more hardware processors in a computing device(s). The term “non-transitory” excludes transitory, propagating signals per se, such as carrier waves or other electromagnetic signals, but includes all forms of physical storage media.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer-readable media comprising instructions which, when executed by one or more hardware processors, cause performance of operations comprising:
accessing a plurality of sets of terminology,
wherein each set of terminology, in the plurality of sets of terminology, comprises a respective plurality of terms;
generating a cross-terminology network that comprises:
(a) a plurality of nodes, each of the plurality of nodes representing at least one term in at least one set of terminology of the plurality of sets of terminology;
(b) an inter-terminology connection between:
(i) a first node, of the plurality of nodes, that represents a first term in a first set of terminology of the plurality of sets of terminology; and
(ii) a second node, of the plurality of nodes, that represents a second term in a second set of terminology of the plurality of sets of terminology; and
(c) an intra-terminology connection between:
(i) a third node, of the plurality of nodes, that represents a third term in the first set of terminology; and
(ii) a fourth node, of the plurality of nodes, that represents a fourth term in the first set of terminology;
receiving a first query associated with the first term;
extracting a subset of the cross-terminology network based on the first term to generate a limited network at least by:
identifying a subset of nodes that are within “N” hops from the first node; and
generating the limited network based on:
the subset of nodes; and
one or more connections of each particular node of the subset of nodes that connects the particular node to at least one other node in the subset of nodes; and
executing the first query based on the terms associated with nodes in the limited network.

2. The one or more non-transitory computer-readable media of claim 1, wherein executing the first query based on the terms associated with the nodes in the limited network comprises:
executing the first query based on definitions, explanations, and/or information indexed using the terms.

3. The one or more non-transitory computer-readable media of claim 1, wherein the operations further comprise:
applying a reasoner to terms within the plurality of sets of terminologies to determine a first relationship between the first term and the second term;
wherein generating the cross-terminology network comprises generating the inter-terminology connection based on the first relationship determined by the reasoner.

4. The one or more non-transitory computer-readable media of claim 3, wherein the operations further comprise:
applying the reasoner to terms within the plurality of sets of terminologies to determine a second relationship between the third term and the fourth term;
wherein generating the cross-terminology network comprises generating the intra-terminology
connection based on the second relationship determined by the reasoner.

5. The one or more non-transitory computer-readable media of claim 1, wherein the operations further comprise:
accessing patient data of a patient associated with the first term;
identifying a subset of the patient data associated with the first term; and
comparing the subset of the patient data with the terms in the limited network to identify overlap between the subset of patient data and the terms in the limited network; and
in response to the query, presenting information based on data that overlaps between the subset of patient data and the terms in the limited network.

6. The one or more non-transitory computer-readable media of claim 1, wherein the operations further comprise:
presenting information from the limited network for display to a user, wherein presenting information from the limited network comprises:
applying a first machine learning model trained to select a template for displaying information from a limited network to the limited network to select a template for displaying the information;
populating the selected template with the information from the limited network to generate a populated template; and
displaying the populated template on a user interface.

7. The one or more non-transitory computer-readable media of claim 1, wherein the operations further comprise:
identifying one or more treatment or intervention codes associated with the first term and
initiating one or more treatment actions based on the one or more treatment or intervention codes.

8. The one or more non-transitory computer-readable media of claim 1, wherein the operations further comprise:
generating query results;
identifying one or more clinical guidelines associated with the first term
determining, based on the one or more clinical guidelines, whether a diagnostic test or treatment procedure is indicated; and
scheduling a medical procedure based on the query result and the one or more guidelines.

9. The one or more non-transitory computer-readable media of claim 1, wherein the operations first query further comprises:
identifying one or more medication terms within the limited network and determining whether a contraindication exists, and
generating a safety alert including evidence supporting the contraindication and one or more recommended alternative treatments.

10. The non-transitory computer-readable media of claim 1, wherein executing the first query further comprises:
generating query results;
determining whether prior authorization is required for a procedure or medication identified in the query results; and
generating and transmitting a prior authorization request to a payer system, the request including clinical justification data derived from nodes of the limited network.

11. A method comprising:
accessing a plurality of sets of terminology,
wherein each set of terminology, in the plurality of sets of terminology, comprises a respective plurality of terms;
generating a cross-terminology network that comprises:
(a) a plurality of nodes, each of the plurality of nodes representing at least one term in at
least one set of terminology of the plurality of sets of terminology;
(b) an inter-terminology connection between:
(iii) a first node, of the plurality of nodes, that represents a first term in a first set of terminology of the plurality of sets of terminology; and
(iv) a second node, of the plurality of nodes, that represents a second term in a second set of terminology of the plurality of sets of terminology; and
(c) an intra-terminology connection between:
(iii) a third node, of the plurality of nodes, that represents a third term in the first set of terminology; and
(iv) a fourth node, of the plurality of nodes, that represents a fourth term in the first set of terminology;
receiving a first query associated with the first term;
extracting a subset of the cross-terminology network based on the first term to generate a limited network at least by:
identifying a subset of nodes that are within “N” hops from the first node; and
generating the limited network based on:
the subset of nodes; and
one or more connections of each particular node of the subset of nodes that connects the particular node to at least one other node in the subset of nodes; and
executing the first query based on the terms associated with nodes in the limited network,
wherein the method is performed by at least one device including a hardware processor.

12. The method of claim 11, wherein executing the first query based on the terms associated with the nodes in the limited network comprises executing the first query based on definitions, explanations, and/or information indexed using the terms.

13. The method of claim 11, further comprising:
applying a reasoner to terms within the plurality of sets of terminologies to determine a first relationship between the first term and the second term;
wherein generating the cross-terminology network comprises generating the inter-terminology connection based on the first relationship determined by the reasoner.

14. The method of claim 13, further comprising:
applying the reasoner to terms within the plurality of sets of terminologies to determine a second relationship between the third term and the fourth term;
wherein generating the cross-terminology network comprises generating the intra-terminology connection based on the second relationship determined by the reasoner.

15. The method of claim 11, further comprising:
accessing patient data of a patient associated with the first term;
identifying a subset of the patient data associated with the first term; and
comparing the subset of the patient data with the terms in the limited network to identify overlap between the subset of patient data and the terms in the limited network; and
in response to the query, presenting information based on data that overlaps between the subset of patient data and the terms in the limited network.

16. The method of claim 11, comprising:
presenting information from the limited network for display to a user, wherein presenting information from the limited network comprises:
applying a first machine learning model trained to select a template for displaying information from a limited network to the limited network to select a template for displaying the information;
populating the selected template with the information from the limited network to generate a populated template; and
displaying the populated template on a user interface.

17. The method of claim 11, further comprising:
identifying one or more treatment or intervention codes associated with the first term; and
initiating one or more treatment actions based on the one or more treatment or intervention codes.

18. The method of claim 11, further comprising:
generating query results;
identifying one or more clinical guidelines associated with the first term;
determining, based on the one or more clinical guidelines, whether a diagnostic test or treatment procedure is indicated; and
scheduling a medical procedure based on the query result and the one or more clinical guideline.

19. The method of claim 11, further comprising:
identifying one or more medication terms within the limited network and determining whether a contraindication exists, and
generating a safety alert including evidence supporting the contraindication and one or more recommended alternative treatments.

20. A system comprising:
at least one device including a hardware processor;
the system being configured to perform operations comprising:
accessing a plurality of sets of terminology,
wherein each set of terminology, in the plurality of sets of terminology, comprises a respective plurality of terms;
generating a cross-terminology network that comprises:
(a) a plurality of nodes, each of the plurality of nodes representing at least one term in at least one set of terminology of the plurality of sets of terminology;
(b) an inter-terminology connection between:
(i) a first node, of the plurality of nodes, that represents a first term in a first set of terminology of the plurality of sets of terminology; and
(ii) a second node, of the plurality of nodes, that represents a second term in a second set of terminology of the plurality of sets of terminology; and
(c) an intra-terminology connection between:
(i) a third node, of the plurality of nodes, that represents a third term in the first set of terminology; and
(ii) a fourth node, of the plurality of nodes, that represents a fourth term in the first set of terminology;
receiving a first query associated with the first term;
extracting a subset of the cross-terminology network based on the first term to generate a limited network at least by:
identifying a subset of nodes that are within “N” hops from the first node; and
generating the limited network based on:
the subset of nodes; and
one or more connections of each particular node of the subset of nodes that connects the particular node to at least one other node in the subset of nodes; and
executing the first query based on the terms associated with nodes in the limited network.
